SEQUENCE LISTING (10 sequences)

SEQ ID NO: 1 - DNA, 20 bp, Artificial Sequence
  human solute carrier family 22, member 1, transcript variant 1 (SLC22A1), organic cation transporter 1 (OCT1, hOCT1) RT-PCR sense primer
ctgtgtagaccccctggcta

SEQ ID NO: 2 - DNA, 20 bp, Artificial Sequence
  human solute carrier family 22, member 21, transcript variant 1 (SLC22A1), organic cation transporter 1 (OCT1, hOCT1) RT-PCR antisense primer
gtgtagccagccatccagtt

SEQ ID NO: 3 - DNA, 20 bp, Artificial Sequence
  human solute carrier family 22, member 2 (SLC22A2), organic cation transporter 2 (OCT2, hOCT2, MGC32628) RT-PCR sense primer
cctggtatgtgccaactcct

SEQ ID NO: 4 - DNA, 20 bp, Artificial Sequence
  human solute carrier family 22, member 2 (SLC22A2), organic cation transporter 2 (OCT2, hOCT2, MGC32628) RT-PCR antisense primer
caccaggagcccaactgtat

SEQ ID NO: 5 - DNA, 20 bp, Artificial Sequence
  human solute carrier family 22, member 3 (SLC22A3), organic cation transporter 3 (OCT3, hOCT3), extraneuronal monoamine transporter (EMT, EMTh) RT-PCR sense primer
atcgtcagcgagtttgacct

SEQ ID NO: 6 - DNA, 20 bp, Artificial Sequence
  human solute carrier family 22, member 3 (SLC22A3), organic cation transporter 3 (OCT3, hOCT3), extraneuronal monoamine transporter (EMT, EMTh) RT-PCR antisense primer
ttgaatcacgattcccacaa

SEQ ID NO: 7 - DNA, 20 bp, Artificial Sequence
  human glyceraldehyde-3-phosphate dehydrogenase (GAPDH, G3PD, GAPD, MGC88685), aging-associated gene 9 protein RT-PCR sense primer
aatcccatcaccatcttcca

SEQ ID NO: 8 - DNA, 20 bp, Artificial Sequence
  human glyceraldehyde-3-phosphate dehydrogenase (GAPDH, G3PD, GAPD, MGC88685), aging-associated gene 9 protein RT-PCR antisense primer
tgtggtcatgagtccttcca

SEQ ID NO: 9 - DNA, 17 bp, Artificial Sequence
  dog glyceraldehyde-3-phosphate dehydrogenase (GAPDH) RT-PCR sense primer
ggtgatgctggtgagta

SEQ ID NO: 10 - DNA, 20 bp, Artificial Sequence
  dog glyceraldehyde-3-phosphate dehydrogenase (GAPDH) RT-PCR antisense primer
gtggaagcagggatgatgtt

(19) United States
(12) Patent Application Publication
(10) Pub. No.: US 2010/0028259 A1
(43) Pub. Date: Feb. 4, 2010

(54) USE OF ORGANIC CATION TRANSPORTERS FOR CANCER DIAGNOSIS AND THERAPY

(75) Inventors: Kathleen M. Giacomini, Atherleen, CA (US); Joe W. Gray, San Francisco, CA (US); Anna Lapuk, Larkspur, CA (US); Shuzhong Zhang, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/296,887

(22) PCT Filed: Apr. 18, 2007
(86) PCT No.: PCT/US07/66848

§ 371 Date: Sep. 30, 2009

Related U.S. Application Data
(60) Provisional application No. 60/793,803, filed on Apr. 20, 2006.

(51) Int. Cl. A61K 49/00; C12Q 1/68; G01N 33/53; A61B 5/05

(52) U.S. Cl. 424/9.1; 435/6; 435/7.21; 600/407

(57) ABSTRACT

The present invention provides, for the first time, the finding that organic cation transporters (OCTs) are major determinants of the anticancer activity of platinum-based drugs such as oxaliplatin, and therefore have clinical significance for selecting oxaliplatin as the preferred therapy for a cancer that expresses one or more OCTs, such as colorectal cancer or liver cancer. In addition, the OCT genotype can also be used to predict oxaliplatin response or to select therapy. The present invention also provides methods of treating or inhibiting cancers that expresses one or more OCTs by administering a therapeutically effective amount of a platinum-based drug such as an oxaliplatin analog having an organic non-leaving group with an increased size. The present invention further provides methods of sensitizing a therapy resistant cancer to a platinum-based drug such as oxaliplatin by administering a therapeutically effective amount of a nucleic acid encoding an OCT. Compositions, kits, and integrated systems for carrying out the diagnostic, prognostic, and therapeutic methods of the present invention are also provided.

### CROSS-REFERENCES TO RELATED APPLICATIONS

The present application claims priority to U.S. Ser. No. 60/793,803, filed Apr. 20, 2006, herein incorporated by reference in its entirety.

### STATEMENT AS TO RIGHTS TO INVENTIONS MADE UNDER FEDERALLY SPONSORED RESEARCH OR DEVELOPMENT

This invention was made with Government support under Grant Nos. GM36780 and GM61390, awarded by the National Institutes of Health. The U.S. Government has certain rights in this invention.

### BACKGROUND OF THE INVENTION

Cancer is the second leading cause of death behind heart disease. In fact, cancer incidence and death figures account for about 10% of the U.S. population in certain areas of the United States (National Cancer Institute's Surveillance, Epidemiology, and End Results (SEER) database and Bureau of the Census statistics; see, Harrison's Principles of Internal Medicine, Kasper et al., 16^{th} ed., 2005, Chapter 66). The five leading causes of cancer deaths among men are lung cancer, prostate cancer, colon and rectum cancer, pancreatic cancer, and leukemia. The five leading causes of cancer deaths among women are lung cancer, breast cancer, colon cancer, ovarian cancer, and pancreatic cancer. When detected at locally advanced or metastatic stages, no consistently curative treatment regimen exists. Treatment for metastatic cancer includes immunotherapy, hormonal ablation, radiation therapy, chemotherapy, hormonal therapy, and combination therapies. Unfortunately, for prostate cancer and hormone dependent tumors, there is frequent relapse of an aggressive androgen independent disease that is insensitive to further hormonal manipulation or to treatment with conventional chemotherapy (Ghosh et al., Proc. Natl. Acad. Sci. U.S.A., 95:13182-13187 (1998)).

Platinum-based drugs are among the most active anticancer agents and cisplatin represents one of the three most widely used cancer chemotherapeutics (Wong et al., Chem. Rev., 99:2451-2466 (1999)). Although cisplatin is effective against a number of solid tumors, especially testicular and ovarian cancer, its clinical use is limited because of its toxic effects as well as the intrinsic and acquired resistance of some tumors to this drug (Weiss et al., Drugs, 46:360-377 (1993)). To overcome these limitations, platinum analogs with lower toxicity and greater activity in cisplatin-resistant tumors have been developed and tested, resulting in the approval of carboplatin and oxaliplatin in the United States. Carboplatin has the advantage of being less nephrotoxic, but its cross-resistance with cisplatin limits its application in otherwise cisplatin-treatable diseases (Weiss et al., supra). Oxaliplatin, however, exhibits a different anticancer spectrum from that of cisplatin (Raymond et al., Ann. Oncol., 9:1053-1071 (1998); Rixe et al., Biochem. Pharmacol., 52:1855-1865 (1996)). It has been approved as the first or second line therapy in combination with 5-fluoruracil/leucovorin for advanced colorectal cancer, for which cisplatin and carboplatin are essentially inactive (Misset et al., Crit. Rev. Oncol. Hematol., 35:75-93 (2000)). In spite of their distinct antitumor specificities, cisplatin and oxaliplatin, as well as other platinum compounds, share similar mechanisms of action. In particular, their cytotoxicity arises primarily from covalent binding to DNA after aquation to form mono- and diaqua complexes (Pinto et al., Biochim. Biophys. Acta, 780:167-180 (1985); Zamble et al., Trends Biochem. Sci., 20:435-439 (1995)). This chemistry initiates a series of biochemical cascades, eventually leading to cell death (Pinto et al., supra; Wang et al., Nat. Rev. Drug Discov., 4:307-320 (2005)).

Because cisplatin and oxaliplatin target similar DNA sites for binding and form similar types of DNA adducts (Jennerwein et al., Chem. Biol. Interact., 70:39-49 (1989); Page et al., Biochemistry, 29:1016-1024 (1990); Woynarowski et al., Mol. Pharmacol., 54:770-777 (1998)), mainly 1,2- and 1,3-intrastrand cross-links involving purine nucleotides, the mechanisms responsible for their distinct tumor specificities may involve events other than their interaction with and binding to DNA. Studies aiming to identify such mechanisms have focused largely on the cellular processing of cisplatin- and oxaliplatin-DNA adducts (Chaney et al., Crit. Rev. Oncol. Hematol., 53:3-11 (2005); Vaisman et al., Biochemistry, 38:11026-11039 (1999)). However, differences in the mechanism(s) controlling the cellular uptake and efflux of these platinum compounds, although rarely investigated, could also be important, since reduced intracellular accumulation is the most common observation in cisplatin-resistant cells (Andrews et al., Cancer Cells, 2:35-43 (1990); Gately et al., Br. J. Cancer, 67:1171-1176 (1993)).

Recent studies suggest a direct involvement of the human copper influx transporter, Ctr1, in the cellular uptake of cisplatin, carboplatin, and oxaliplatin to a varying extent (Song et al., Mol. Cancer. Ther., 3:1543-1549 (2004)). Studies in tumor cell lines indicate, however, that Ctr1 may not affect the formation and corresponding cytotoxicity of cisplatin-DNA adducts (Holzer et al., Mol. Pharmacol., 66:817-823 (2004)). The human copper efflux transporters, ATP7B and ATP7A, also recognize these platinum compounds (Komatsu et al., Cancer Res., 60:1312-1316 (2000); Samimi et al., Mol. Pharmacol., 66:25-32 (2004); Samimi et al., Clin. Cancer Res., 10:4661-4669 (2004)) and their elevated expression has been associated with cisplatin resistance (Aida et al., Gynecol. Oncol., 97:41-45 (2005); Miyashita et al., Oral Oncol., 39:157-162 (2003); Nakayama et al., Clin. Cancer Res., 10:2804-2811 (2004); Samimi et al., Clin. Cancer Res., 9:5853-5859 (2003)). The importance of these interactions in modulating the differential activity and tumor specificity of the platinum compounds is currently unknown.

The organic cation transporters (OCTs), OCT1 (Grundemann et al., Nature, 372:549-552 (1994)), OCT2 (Okuda et al., Biochem. Biophys. Res. Comm., 224:500-507 (1996)), and OCT3 (Kekuda et al., J. Biol. Chem., 273:15971-15979 (1998); Wu et al., J. Biol. Chem., 273:32776-32786 (1998)), are in the class of plasma membrane transporters belonging to the solute carrier (SLC) 22A family. The OCTs mediate intracellular uptake of a broad range of structurally diverse organic cations with molecular weights generally lower than 400 Da (Jonker et al., J. Pharmacol. Exp. Ther., 308:2-9 (2004); Wright, Toxicol. Appl. Pharmacol., 204:309-319 (2005)). Substrates of OCTs include endogenous compounds such as choline, creatinine, and monoamine neurotransmitters, and a variety of xenobiotics such as tetraethylammonium (TEA, a prototypic organic cation), 1-methyl-4-phenylpyridinium (MPP+, a neurotoxin) and clinically used drugs such as metformin, cimetidine, and amantadine (Jonker et al., supra). In humans, OCT1 is primarily expressed in the liver (Gorboulev et al., DNA Cell Biol., 16:871-881 (1997); Zhang et al., Mol. Pharmacol., 51:913-921 (1997); Wright, supra) and less so in the intestine (Muller et al., Biochem. Pharmacol., 70:1851-1860 (2005)), whereas OCT2 is predominantly expressed in the kidney (Gorboulev et al., supra; Wright, supra). OCT3 is expressed in many tissues including placenta, heart, liver, and skeletal muscle (Grundemann et al., Nat. Neurosci., 1:349-351 (1998); Verhaagh et al., Genomics, 55:209-218 (1999)). The expression of the OCTs has also been detected in a number of human cancer cell lines (Hayer-Zillgen et al., Br. J. Pharmacol., 136:829-836 (2002)). The interaction of cisplatin with human OCTs has been investigated and the results are discordant (Briz et al., Mol. Pharmacol., 61:853-860 (2002); Ciarimboli et al., Am. J. Pathol., 167:1477-1484 (2005)). Previous studies indicate that cisplatin is not a substrate of human OCT1 or OCT2 (Briz et al., supra), whereas more recent work indicates that the drug interacts with human and rat OCT2 but not OCT1 (Ciarimboli et al., supra; Yonezawa et al., Biochem. Pharmacol., 70:1823-1831 (2005)). It is not known whether oxaliplatin or carboplatin interacts with these transporters, however, or whether such interactions contribute to their cytotoxicities and differential tumor specificities.

As a result, there is a need in the art for a better understanding of the role of OCTs in mediating the cytotoxic and chemoresistant effects of platinum-based drugs such as oxaliplatin, carboplatin, and cisplatin. There is also a need in the art for methods of diagnosing, providing a prognosis for, and treating cancers such as colorectal cancer and liver cancer based upon the level of OCT expression. The present invention satisfies these and other needs.

### BRIEF SUMMARY OF THE INVENTION

The present invention provides, for the first time, the finding that organic cation transporters (OCTs) are major determinants of the anticancer activity of platinum-based drugs such as oxaliplatin. Therefore, determining whether a cancer expresses an OCT has clinical significance, as it provides a means for selecting oxaliplatin or another OCT platinum substrate as the preferred cancer therapy, i.e., providing a prognosis for oxaliplatin therapy in a subject or acting as a diagnostic marker for selecting oxaliplatin therapy. Such cancers include, e.g., colorectal cancer or liver cancer. For cancers that do not express an OCT, oxaliplatin may not be the preferred therapy, and other courses of treatment may be investigated. The present invention also provides means of selecting the preferred therapy and providing a prognosis for therapy by determining the OCT genotype expressed by the cancer. The present invention also provides methods of treating or inhibiting cancers that expresses one or more OCTs by administering a therapeutically effective amount of a platinum-based drug such as an oxaliplatin analog that has a non-leaving group with an organic component having increased size. In one embodiment, the oxaliplatin analog is linked to a radioactive isotope. The present invention further provides methods of sensitizing a therapy resistant cancer to a platinum-based drug such as oxaliplatin by administering a therapeutically effective amount of a nucleic acid encoding an OCT. Compositions, kits, and integrated systems for carrying out the diagnostic, prognostic, and therapeutic methods of the present invention are also provided.

In one aspect, the present invention provides a method of providing a prognosis for oxaliplatin cancer therapy in a subject or determining if a subject should receive oxaliplatin therapy, the method comprising the steps of:

(a) contacting a sample from the subject with an antibody that specifically binds to OCT protein; and

(b) determining whether or not OCT protein is expressed in the sample, thereby providing a prognosis for oxaliplatin cancer therapy.

In another aspect, the present invention provides a method of providing a prognosis for oxaliplatin cancer therapy in a subject or determining if a subject should receive oxaliplatin therapy, the method comprising the steps of:

(a) contacting a sample from the subject with a primer set of a first oligonucleotide and a second oligonucleotide that each specifically hybridize to an OCT nucleic acid;

(b) amplifying the OCT nucleic acid in the sample; and

(c) determining whether or not the OCT nucleic acid in the sample is expressed in the sample, thereby providing a prognosis for oxaliplatin cancer therapy.

In yet another aspect, the present invention provides a method of providing a prognosis for oxaliplatin cancer therapy in a subject by determining the genotype of an OCT gene, the method comprising the steps of:

(a) contacting a sample from the subject with an antibody that specifically binds to an OCT protein encoded by a selected allele; and

(b) determining whether or not the OCT protein encoded by a selected allele is expressed in the sample, thereby providing a prognosis for the cancer that expresses the OCT.

In still yet another aspect, the present invention provides a method of providing a prognosis for oxaliplatin cancer therapy in a subject by determining the genotype of an OCT gene, the method comprising the steps of:

(a) contacting a sample from the subject with a primer set of a first oligonucleotide and a second oligonucleotide that each specifically hybridize to an OCT allele;

(b) determining whether or not the OCT allele is expressed in the sample, thereby providing a prognosis for oxaliplatin cancer therapy by determining the OCT genotype. In one embodiment, the OCT genotype is selected from the group consisting of: wild-type OCT, G401S, 420 del, S14F, R61c, G220V, V408M, and G465R. In one embodiment, presence of the wild-type or V408M variant predicts a better response to oxaliplatin therapy than the presence of the other variants.

Generally, the methods find particular use in providing a prognosis for oxaliplatin therapy for a cancer including colorectal cancer, liver cancer (i.e., hepatocarcinoma), prostate cancer, renal cancer, bladder cancer, ovarian cancer, breast cancer, lung cancer, leukemia, B-cell lymphoma (e.g., non-Hodgkin's lymphoma, including Burkitt's, Small Cell, and Large Cell lymphomas), and multiple myeloma. Preferably, the cancer that expresses OCT protein or nucleic acid is colorectal cancer or liver cancer.

The present invention also provides a method of localizing a cancer that expresses an OCT in vivo, the method comprising the step of imaging in a subject a cell expressing the OCT (e.g., protein and/or RNA), thereby localizing the cancer in vivo.

In addition, the present invention provides a method of treating or inhibiting a cancer that expresses an OCT, the method comprising the step of administering to a subject in need thereof a therapeutically effective amount of a platinum-based drug that is an oxaliplatin analog comprising a non-leaving group with an organic component having increased size. In one embodiment, the platinum-based drug is selected from the group consisting of oxaliplatin, cisplatin, carboplatin, [Pt(NH3)2(trans-1,2-(OCO)2C6H10], [Pt(Cl2)(en)], cis-[Pt(NH3)(Cy)Cl2], [Pt(S,S-DACH)oxalato], [Pt(R,R-DACH)Cl2], [Pt(S,S-DACH)Cl2], spiroplatin, iproplatin, satraplatin, pharmaceutically acceptable salts thereof, stereoisomers thereof, derivatives thereof, analogs thereof, and combinations thereof. In one embodiment, the oxaliplatin analog is linked to a radioactive isotope.

The platinum-based drug can be administered alone or co-administered (e.g., concurrently or sequentially) in combination therapy with conventionally used chemotherapy, radiation therapy, hormonal therapy, and/or immunotherapy. The methods find particular use in treating colorectal cancer, liver cancer, prostate cancer, renal cancer, bladder cancer, ovarian cancer, breast cancer, lung cancer, leukemia, B-cell lymphoma (e.g., non-Hodgkin's lymphoma, including Burkitt's, Small Cell, and Large Cell lymphomas), multiple myeloma, or other cancers that express one or more of the OCTs described herein.

The present invention further provides a method of sensitizing a therapy resistant cancer to a platinum-based drug, the method comprising the step of administering to a subject in need thereof a therapeutically effective amount of a nucleic acid encoding an OCT.

The methods find particular use in sensitizing tumors that have low or undetectable expression of one or more OCTs to chemotherapy with platinum-based drugs such as oxaliplatin. In certain instances, the OCT nucleic acid and platinum-based drug are co-administered (e.g., concurrently or sequentially) in combination therapy with conventionally used chemotherapy, radiation therapy, hormonal therapy, and/or immunotherapy.

Other objects, features, and advantages of the present invention will be apparent to one of skill in the art from the following detailed description and figures.

### BRIEF DESCRIPTION OF THE DRAWINGS

FIG. 1 shows the expression of human OCT1, OCT2, and OCT3 in stably transfected cell lines. (A) The expression of human OCT1, OCT2, or OCT3 was determined in the cell lines stably transfected with human OCTs (MDCK-hOCT1, HEK-hOCT2, or HEK-hOCT3) or the corresponding empty vectors (MOCK cells) by RT-PCR as described in Example 1. The dog and human GAPDH were used as the expression control for the transfected MDCK and HEK 293 cells, respectively. Lanes 1-6: MDCK-MOCK, MDCK-hOCT1, HEK-MOCK, HEK-hOCT2, HEK-MOCK, and HEK-hOCT3. (B) The cellular uptake of model substrates (TEA for human OCT1 and OCT2; MPP⁺ for human OCT3) was determined in OCT-transfected cells (solid bars) and in the corresponding MOCK cells (open bars). Disopyramide (120 μM) was used as an inhibitor for human OCT1 and cimetidine (1.5 mM) was used as an inhibitor for human OCT2 and OCT3. Data are expressed as mean±SD of six measurements.

FIG. 2 shows the cytotoxicity of oxaliplatin in cells stably transfected with human OCTs. The cytotoxicity of oxaliplatin in (A) OCT1-, (B) OCT2-, and (C) OCT3-transfected cells (open circles) and in the corresponding MOCK cells (solid circles) was determined as described in Example 1. Cells were seeded in 96-well plates at a density of 5,000 cells/well for the transfected MDCK cells and 12,000 cells/well for the transfected HEK 293 cells and exposed to the test compounds for 7 hours on the following day. After a total of 72 hours, cell growth was determined by an MTT assay. In addition, the cytotoxicity of oxaliplatin in (D) OCT1- and (E) OCT2-transfected cells (open symbols) and in the corresponding empty vector-transfected cells (MOCK cells) (solid symbols) in the presence (squares) or absence (circles) of an OCT inhibitor (disopyramide for OCT1, and cimetidine for OCT2) was also simultaneously determined in a similar fashion. When the OCT inhibitors were used, disopyramide (150 μM) or cimetidine (1.5 mM) was added to the incubation medium immediately before the addition of oxaliplatin. The lines represent the predicted data obtained by fitting the observed data using WinNonlin as described in Example 1. Presented are the data from a typical experiment. Three to six independent experiments were performed and similar results were obtained. For clarity, the standard deviation bars in panel D and E were eliminated.

FIG. 3 shows the cellular accumulation rates of platinum after 2-hr exposure to cisplatin, carboplatin, and oxaliplatin. The cellular accumulation rates of platinum in (A) OCT1-, (B) OCT2-, and (C) OCT3-transfected cells and in the corresponding MOCK cells after incubation with cisplatin, carboplatin and oxaliplatin in the presence (open bars) and absence (solid bars) of an OCT inhibitor (disopyramide for OCT1, cimetidine for OCT2 and OCT3) were determined as described in Example 1. (A) MDCK cells were incubated in the antibiotic-free medium containing cisplatin (3 μM), carboplatin (15 μM), or oxaliplatin (3 μM) at 37° C. and 5% CO₂ for 2 hours. For the inhibitor studies, the incubation medium also contained disopyramide (150 μM). (B) HEK 293 cells were incubated in the antibiotic-free medium containing cisplatin (0.3 μM), carboplatin (10 μM), or oxaliplatin (0.3 μM) at 37° C. and 5% CO₂ for 2 hours. For the inhibitor studies, the incubation medium also contained cimetidine (1.5 mM). (C) The study was performed similarly as (B) except that the concentrations of cisplatin, carboplatin, and oxaliplatin in the incubation medium were 2 μM, μM, and 2 μM, respectively. After drug exposure, the cells were washed with ice-cold PBS three times and harvested by scraping and centrifugation. The cell-associated platinum was determined by ICP-MS and normalized for protein content. Data are expressed as mean±SD from a single experiment performed in triplicate. Experiments were replicated for OCT1 and OCT2, and similar results were obtained.

FIG. 4 shows the platinum-DNA adduct formation after 2-hr exposure to oxaliplatin. The content of platinum bound to DNA after 2-hr exposure to oxaliplatin in the presence (open bars) or absence (solid bars) of an OCT inhibitor (disopyramide for OCT1, cimetidine for OCT2) was determined as described in Example 1. (A) Transfected MDCK cells were incubated in the antibiotic-free medium containing oxaliplatin (10 μM) with or without disopyramide (150 μM). (B) Transfected HEK 293 cells were incubated in the antibiotic-free medium containing oxaliplatin (0.6 μM) with or without cimetidine (1.5 mM). After incubation at 37° C. and 5% CO₂ for 2 hours, the cells were washed with ice-cold PBS three times and harvested. The genomic DNA was isolated from the cells and the platinum content associated with DNA was determined by ICP-MS and normalized for DNA content. Data are expressed as mean±SD from a typical experiment performed in triplicate. Two independent experiments were conducted and similar results were obtained.

FIG. 5 shows the chemical structures of the platinum-based compounds used in the present invention.

FIG. 6 shows the platinum-DNA adduct formation after incubation with oxaliplatin or [Pt(R,R-DACH)(H₂O)₂]²⁺ in PB—Cl or PB—SO₄ buffer. Transfected MDCK cells were incubated with oxaliplatin (20 μM) or [Pt(R,R-DACH)(H₂O)₂]²⁺ (1 μM) in PB—Cl or PB—SO₄ buffer at 37° C. and 5% CO₂ for 25 min. Oxaliplatin was freshly prepared and was added to PB—SO₄ buffer immediately, and to PB—Cl buffer half an hour before cell incubation. After incubation, the cells were washed with ice-cold PBS three times and harvested. Genomic DNA was isolated from the harvested cells and the DNA-associated platinum content was determined by ICP-MS and normalized for the DNA content. Data are expressed as mean±SD of three measurements.

FIG. 7 shows the expression of OCT1 and OCT2 in colon cancer cell lines and colon tissue samples. Total RNA was isolated from colon cancer cells and normal or cancerous colon tissues. The expression of OCT1 and OCT2 in these samples was detected by RT-PCR as described in Example 1. A PCR cycle number of 40 was used in all the samples. Human GAPDH expression was used as a loading control and a PCR cycle number of 30 was used for its amplification.

FIG. 8 shows data correlating OCT genotype with reactivity to oxaliplatin. The data shows that the presence of wild-type OCT1 and V408M OCT1 provide the best response to oxaliplatin therapy, as compared to the other variants listed in this figure.

### DETAILED DESCRIPTION OF THE INVENTION

### I. Introduction

Platinum-based drugs such as oxaliplatin, cisplatin, and carboplatin are among the most active anticancer drugs to be approved for clinical use in the United States. In general, oxaliplatin has comparable or superior anticancer activity to cisplatin and higher activity than carboplatin. In addition, oxaliplatin and carboplatin have little or much lower nephrotoxicity, which is a dose-limiting toxicity for cisplatin. However, there is currently a lack of understanding as to the mechanism(s) by which these platinum-based drugs enter cancer cells to bind DNA and trigger cell cycle arrest and apoptosis. There is also a lack of understanding as to the mechanism(s) by which cancer cells become resistant to these platinum-based drugs.

The present invention is based, in part, on the surprising discovery that organic cation transporters (OCTs) such as OCT1 and OCT2 are major determinants of the anticancer activity of oxaliplatin and contribute to differences in the tumor specificities of platinum-based drugs. In particular, the present invention illustrates that tumors expressing OCTs have increased sensitivity to oxaliplatin cytotoxicity. As a result, the use of OCTs as markers for selecting specific platinum-based drugs is advantageous for individualizing cancer therapy. As a non-limiting example, the expression level of one or more OCTs in a tumor can be used to determine the sensitivity and/or chemoresistance of the tumor to a specific platinum-based drug such as oxaliplatin. In addition, the development of new anticancer drugs that are specifically targeted to OCTs based upon the structure-activity studies described herein represents a novel strategy for targeted drug therapy.

Detection of OCT expression (e.g., OCT1, OCT2, and/or OCT3 expression) is particularly useful as an indicator for oxaliplatin therapy for cancers such as colorectal cancer, liver cancer, prostate cancer, renal cancer, bladder cancer, ovarian cancer, breast cancer, lung cancer, leukemia, B-cell lymphoma (e.g., non-Hodgkin's lymphoma, including Burkitt's, Small Cell, and Large Cell lymphomas), and multiple myeloma. Detection can include, for example, the level of OCT mRNA or protein expression, or the localization (e.g., nuclear, cytoplasmic, cell surface, etc.) of OCT mRNA or protein. Expression of OCT can be examined in whole cell or tissue samples. In terms of early diagnosis, treatment decisions, and prognosis, needle, surgical, or bone marrow biopsies can be used and examined by techniques such as immunoblotting or immunohistochemistry and compared to control cells or tissue, e.g., from a healthy subject. In addition, microlaser microdissection can be used to isolate a few cells and run RT-PCR for OCT nucleic acid. The following PCR primers can be used to detect OCT1 nucleic acid: (sense, SEQ ID NO:1) 5′-CTG TGT AGA CCC CCT GGC TA-3′; and (antisense, SEQ ID NO:2) 5′-GTG TAG CCA GCC ATC CAG TT-3′. The following PCR primers can be used to detect OCT2 nucleic acid: (sense, SEQ ID NO:3) 5′-CCT GGT ATG TGC CAA CTC CT-3′; and (antisense, SEQ ID NO:4) 5′-CAC CAG GAG CCC AAC TGT AT-3′. The following PCR primers can be used to detect OCT3 nucleic acid: (sense, SEQ ID NO:5) 5′-ATC GTC AGC GAG TTT GAC CT-3′; and (antisense, SEQ ID NO:6) 5′-TTG AAT CAC GAT TCC CAC AA-3′.

In determining the levels of protein expression or the localization of OCT protein, polyclonal or monoclonal antibodies that specifically bind to OCT1, OCT2, or OCT3 can be used.

In one embodiment, the methods of the present invention are used in providing a prognosis for oxaliplatin therapy for a colorectal cancer or a subtype thereof, e.g., colorectal adenocarcinoma (i.e., mucinous, signet ring cell), colorectal sarcoma, colorectal melanoma, colorectal carcinoid, or colorectal lymphoma. The methods of the present invention are also useful in providing a prognosis for oxaliplatin therapy for liver cancer or a subtype thereof, e.g., fibrolamellar hepatocarcinoma, cholangiocarcinoma, angiosarcoma, hemangiosarcoma, or hepatoblastoma. In carrying out the prognostic methods described herein, the determination of whether or not OCT protein or nucleic acid is expressed can be made, e.g., by comparing a test sample to a control autologous sample from normal tissue.

In carrying out the prognostic methods of the present invention, the sample can be taken from a tissue of a primary tumor or a metastatic tumor. A tissue sample can be taken, for example, by an excisional biopsy, an incisional biopsy, a needle biopsy, a surgical biopsy, a bone marrow biopsy, or any other biopsy technique known in the art. In some embodiments, the tissue sample is microlaser microdissected cells from a needle biopsy. In other embodiments, the tissue sample is a metastatic cancer tissue sample. In yet other embodiments, the tissue sample is fixed, e.g., with paraformaldehyde, and embedded, e.g., in paraffin. Suitable tissue samples can be obtained from colon, rectum, liver, kidney, bladder, prostate, ovary, lung, breast, etc., as well as from the blood, serum, saliva, urine, bone, lymph node, or other tissue.

In certain instances, the diagnostic or prognostic methods of the present invention further comprise genotyping the subject to determine an OCT genotype, i.e., determining the presence or absence of a variant allele at a polymorphic site in the OCT1, OCT2, and/or OCT3 gene. In one embodiment, the OCT genotype is selected from the group consisting of: wild-type OCT, G401S, 420 del, S14F, R61c, G220V, V408M, and G465R. In one embodiment, presence of the wild-type or V408M variant predicts a better response to oxaliplatin therapy.

The present invention also provides a method of localizing a cancer that expresses an OCT (e.g., OCT1, OCT2, and/or OCT3) in vivo, the method comprising the step of imaging in a subject a cell expressing the OCT (e.g., protein and/or RNA), thereby localizing the cancer in vivo.

In addition, the present invention provides a method of treating or inhibiting a cancer that expresses an OCT (e.g., OCT1, OCT2, and/or OCT3), the method comprising the step of administering to a subject in need thereof a therapeutically effective amount of a platinum-based drug.

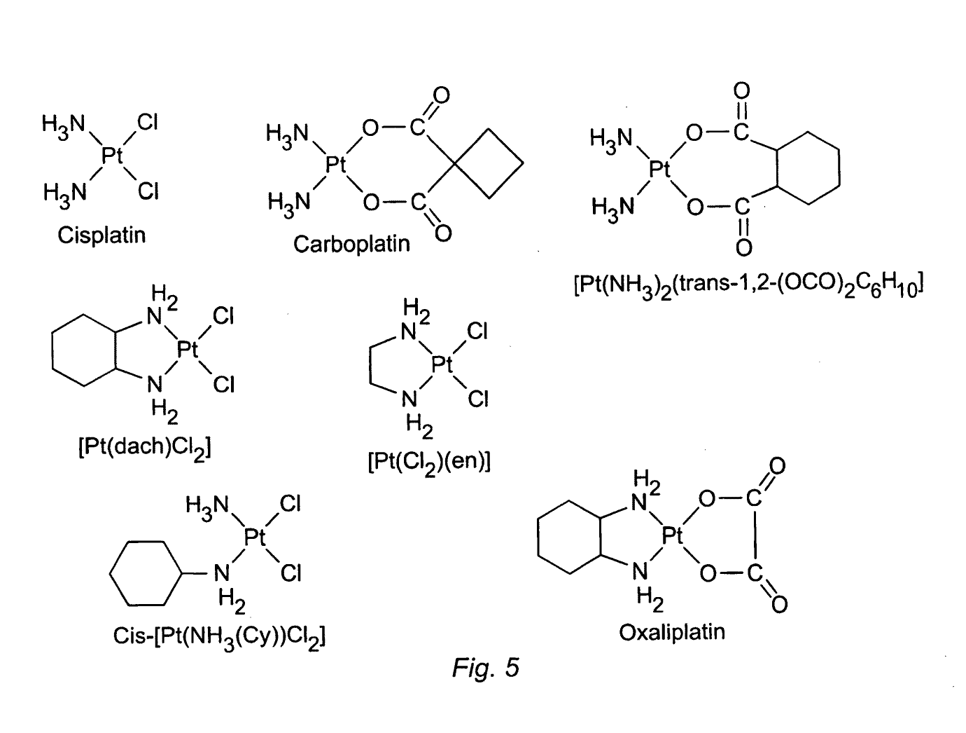

In carrying out the therapeutic methods of the present invention, the platinum-based drug is an oxaliplatin analog that has a non-leaving group with an organic component having increased size. In one embodiment, the drug can be selected from the group consisting of oxaliplatin, cisplatin, carboplatin, [Pt(NH₃)₂(trans-1,2-(OCO)₂C₆H₁₀], [Pt(Cl₂)(en)], cis-[Pt(NH₃)(Cy)Cl₂], [Pt(S,S-DACH)oxalato], [Pt(R,R-DACH)Cl₂], [Pt(S,S-DACH)Cl₂], spiroplatin, iproplatin, satraplatin, pharmaceutically acceptable salts thereof, stereoisomers thereof, derivatives thereof, analogs thereof, and combinations thereof. FIG. 5 provides the chemical structures of many of these platinum-based drugs. The oxaliplatin analog is optionally linked to a radioisotope such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi.

In certain instances, the platinum-based drug is co-administered with an additional cancer therapy. For example, the platinum-based drug can be co-administered (e.g., concurrently or sequentially) in combination therapy with conventionally used chemotherapy, radiation therapy, hormonal therapy, and/or immunotherapy. The methods find particular use in treating colorectal cancer, liver cancer, prostate cancer, renal cancer, bladder cancer, ovarian cancer, breast cancer, lung cancer, leukemia, B-cell lymphoma (e.g., non-Hodgkin's lymphoma, including Burkitt's, Small Cell, and Large Cell lymphomas), multiple myeloma, or other cancers that express one or more of the OCTs described herein.

The present invention further provides a method of sensitizing a therapy resistant cancer to a platinum-based drug, the method comprising the step of administering to a subject in need thereof a therapeutically effective amount of a nucleic acid encoding an OCT (e.g., OCT1, OCT2, and/or OCT3).

The methods find particular use in sensitizing tumors that have low or undetectable expression of one or more OCTs to chemotherapy with platinum-based drugs such as those described herein (e.g., oxaliplatin) by increasing the level of OCT expression in the tumor. In certain instances, the OCT nucleic acid is co-administered (e.g., concurrently or sequentially) in combination with platinum-based drugs and/or conventionally used chemotherapy, radiation therapy, hormonal therapy, and/or immunotherapy.

### II. Definitions

As used herein, the following terms have the meanings ascribed to them unless specified otherwise.

“Organic cation transporter” or “OCT” refers to nucleic acids (e.g., gene, pre-mRNA, mRNA), polypeptides, polymorphic variants, alleles, mutants, and interspecies homologs that: (1) have an amino acid sequence that has greater than about 60% amino acid sequence identity, e.g., about 65%, 70%, 75%, 80%, 85%, 90%, 95%, preferably about 91%, 92%, 93%, 94%, 95%, 96%, 97%, 98%, 99%, or greater amino acid sequence identity, preferably over a region of at least about 25, 50, 100, 200, 500, 1000, or more amino acids, to a polypeptide encoded by a referenced nucleic acid or an amino acid sequence described herein, including OCT1, OCT2, and OCT3; (2) specifically bind to antibodies (e.g., polyclonal antibodies) raised against an immunogen comprising a referenced amino acid sequence, immunogenic fragments thereof, and conservatively modified variants thereof, including OCT1, OCT2, and OCT3; (3) specifically hybridize under stringent hybridization conditions to a nucleic acid encoding a referenced amino acid sequence, and conservatively modified variants thereof, including a nucleic acid encoding OCT1, OCT2, or OCT3; and/or (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence, including a reference nucleic acid encoding OCT1, OCT2, or OCT3. A polynucleotide or polypeptide sequence is typically from a mammal including, but not limited to, primate (e.g., human), rodent (e.g., rat, mouse, hamster), cow, pig, horse, sheep, or any mammal. The nucleic acids and proteins of the present invention include both naturally-occurring and recombinant molecules. An exemplary human nucleic acid encoding OCT1 is provided by Accession No. NM_{—}003057; exemplary protein sequences are provided by Accession Nos. NP_{—}003048 and NP_{—}694857. An exemplary human nucleic acid encoding OCT2 is provided by Accession No. NM_{—}003058; exemplary protein sequences are provided by Accession Nos. NP_{—}003049 and NP_{—}694861. An exemplary human nucleic acid encoding OCT3 is provided by Accession No. NM_{—}021977; exemplary protein sequences are provided by Accession Nos. NP_{—}068812 and 075751. Truncated, alternatively spliced, precursor, and mature forms of OCTs are also included in the foregoing definition.

The term “cancer” refers to human cancers and carcinomas, sarcomas, adenocarcinomas, lymphomas, leukemias, solid and lymphoid cancers, etc. Examples of different types of cancer include, but are not limited to, colorectal cancer, liver cancer (i.e., hepatocarcinoma), prostate cancer, renal cancer (i.e., renal cell carcinoma), bladder cancer, lung cancer (e.g., non-small cell lung cancer), breast cancer, thyroid cancer, pleural cancer, pancreatic cancer, ovarian cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. In preferred embodiments, the methods of the present invention are useful for diagnosing, proving a prognosis for, and treating colorectal cancer, liver cancer, or a subtype thereof.

The term “platinum-based drug (or compound)” as used herein refers to a compound comprising a heavy metal complex containing a central atom of platinum surrounded by organic and/or inorganic functionalities. Non-limiting examples of platinum-based drugs include oxaliplatin, cisplatin, carboplatin, [Pt(NH₃)₂(trans-1,2-(OCO)₂C₆H₁₀], [Pt(Cl₂)(en)], cis-[Pt(NH₃)(Cy)Cl₂], [Pt(S,S-DACH)oxalato], [Pt(R,R-DACH)Cl₂], [Pt(S,S-DACH)Cl₂], spiroplatin, iproplatin, satraplatin, pharmaceutically acceptable salts thereof, stereoisomers thereof, derivatives thereof, analogs thereof, and combinations thereof. FIG. 5 illustrates various platinum-based drugs suitable for use in the therapeutic methods of the present invention. The term also applies to oxaliplatin analogs having a non-leaving group with an organic component having increased size.

The term “expression” refers to a gene that is transcribed or translated at a detectable level. As used herein, expression also encompasses “overexpression,” which refers to a gene that is transcribed or translated at a detectably greater level, usually in a cancer cell, in comparison to a normal cell. Expression therefore refers to both expression of OCT protein and RNA, as well as local overexpression due to altered protein trafficking patterns and/or augmented functional activity. Expression can be detected using conventional techniques for detecting protein (e.g., ELISA, Western blotting, flow cytometry, immunofluorescence, immunohistochemistry, etc.) or mRNA (e.g., RT-PCR, PCR, hybridization, etc.). One skilled in the art will know of other techniques suitable for detecting expression of OCT protein or mRNA. Cancerous cells, e.g., cancerous colon or liver cells, can express (e.g., overexpress) one or more OCTs (e.g., OCT1, OCT2, and/or OCT3) at a level of at least about 5%, 10%, 15%, 20%, 25%, 30%, 35%, 40%, 45%, 50%, 55%, 60%, 65%, 70%, 75%, 80%, 85%, 90%, or 95% in comparison to normal, non-cancerous cells such as colon or liver cells. Cancerous cells can also have at least about a 1-fold, 2-fold, 3-fold, 4-fold, 5-fold, 6-fold, or 7-fold higher level of OCT transcription or translation in comparison to normal, non-cancerous cells. In certain instances, the cancer cell sample is autologous.

“Therapy resistant” cancers, tumor cells, and tumors refer to cancers that have become resistant to both apoptosis-mediated (e.g., through death receptor cell signaling, for example, Fas ligand receptor, TRAIL receptors, TNF-R1, chemotherapeutic drugs, radiation, etc.) and non-apoptosis mediated (e.g., toxic drugs, chemicals, etc.) cancer therapies including, but not limited to, chemotherapy, hormonal therapy, radiotherapy, immunotherapy, and combinations thereof.

“Therapeutic treatment” and “cancer therapies” refers to apoptosis-mediated and non-apoptosis mediated cancer therapies including, without limitation, chemotherapy, hormonal therapy, radiotherapy, immunotherapy, and combinations thereof.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “marker” refers to any biochemical marker, serological marker, genetic marker, or other clinical or echographic characteristic that can be used to diagnose or provide a prognosis for a cancer that expresses at least one OCT according to the methods of the present invention. Preferably, the marker is an OCT protein or nucleic acid marker such as an OCT1, OCT2, and/or OCT3 marker.

The term “sample” includes sections of tissues such as biopsy and autopsy samples, and frozen sections taken for histological purposes. Such samples include blood and blood fractions or products (e.g., serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., colon, prostate, kidney, bladder, lymph node, liver, bone marrow, blood cell, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably about 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithm with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or over a region that is about 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from about 20 to about 600, usually from about 50 to about 200, more usually from about 100 to about 150, in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithms suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the present invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length of 3, and expectation (E) of 10, and the BLOSUM62 scoring matrix (see, Henikoff et al., Proc. Natl. Acad. Sci. USA, 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally-occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of OCT. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′-end or at the 3′-end. Polypeptides can be truncated at the N-terminal end or the C-terminal end. Truncated versions of nucleic acid or polypeptide sequences can be naturally-occurring or recombinantly created.

“Polymorphism” refers to the occurrence of two or more genetically determined alternative sequences or alleles in a population (e.g., OCT1, OCT2, or OCT3 alleles). A “polymorphic site” refers to the locus at which divergence occurs. Preferred polymorphic sites have at least two alleles, each occurring at frequency of greater than 1%, and more preferably greater than 10% or 20% of a selected population. A polymorphic locus can be as small as one base pair (single nucleotide polymorphism, or SNP). Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allele is arbitrarily designated as the reference allele and other alleles are designated as alternative or “variant alleles.” The allele occurring most frequently in a selected population is sometimes referred to as the “wild-type” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest.

The term “genotype” as used herein broadly refers to the genetic composition of an organism, including, for example, whether a diploid organism is heterozygous or homozygous for one or more variant alleles of interest.

The term “gene amplification” refers to a cellular process characterized by the production of multiple copies of any particular piece of DNA. For example, a tumor cell amplifies, or copies, chromosomal segments naturally as a result of cell signals and sometimes environmental events. The process of gene amplification leads to the production of many copies of the genes that are located on that region of the chromosome. In certain instances, so many copies of the amplified region are produced that they can form their own small pseudo-chromosomes called double-minute chromosomes. The genes on each of the copies can be transcribed and translated, leading to an overproduction of the mRNA and protein corresponding to the amplified genes.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally-occurring amino acid, as well as to naturally-occurring amino acid polymers and non-naturally occurring amino acid polymers.

The term “amino acid” refers to naturally occurring and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, and methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid.

Amino acids may be referred to herein by either their commonly known three-letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill in the art will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or “detectable moiety” refers to a composition detectable by spectroscopic, photochemical, biochemical, immunochemical, chemical, or other physical means. For example, a detectable moiety can be coupled either directly or indirectly to the anti-OCT antibodies described herein using methods well known in the art. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, electron-dense reagents, biotin, digoxigenin, haptens, and the like.

The term “recombinant,” when used with reference, e.g., to a cell, nucleic acid, protein, or vector, indicates that the cell, nucleic acid, protein, or vector has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, overexpressed, underexpressed, or not expressed at all.

The term “heterologous,” when used with reference to portions of a nucleic acid, indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and may be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably at least ten times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill in the art will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and about 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of about 90-95° C. for about 30 sec-2 min., an annealing phase lasting about 30 sec.-2 min., and an extension phase of about 72° C. for about 1-2 min. Protocols and guidelines for low and high stringency amplification reactions are provided, e.g., in Innis et al. (1990) PCR Protocols, A Guide to Methods and Applications, Academic Press, Inc. N.Y.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to about 110 or more amino acids primarily responsible for antigen recognition. The terms variable light chain (V_{L}) and variable heavy chain (V_{H}) refer to these light and heavy chains, respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology, Paul ed., 3d ed. 1993). While various antibody fragments are defined in terms of the digestion of an intact antibody, one of skill in the art will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, any technique known in the art can be used (see, e.g., Kohler and Milstein, Nature, 256:495-497 (1975); Kozbor et al., Immunology Today, 4: 72 (1983); Cole et al., pp. 77-96 in Monoclonal Antibodies and Cancer Therapy, Alan R. Liss, Inc. (1985); Coligan, Current Protocols in Immunology (1991); Harlow and Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (see, e.g., U.S. Pat. Nos. 4,946,778 and 4,816,567) can be adapted to produce antibodies to the polypeptides of the present invention. Also, transgenic mice or other organisms such as other mammals may be used to express humanized or human antibodies (see, e.g., U.S. Pat. Nos. 5,545,807; 5,545,806; 5,569,825; 5,625,126; 5,633,425; and 5,661,016, Marks et al., Biotechnology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg et al., Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., supra). Antibodies can also be made bispecific, i.e., able to recognize two different antigens (see, e.g., PCT Patent Publication No. WO 93/08829; Traunecker et al., EMBO J, 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980, PCT Patent Publication Nos. WO 91/00360 and WO 92/200373; and EP Patent No. 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature, 321:522-525 (1986); Riechmann et al., Nature, 332:323-327 (1988); Verhoeyen et al., Science, 239:1534-1536 (1988) and Presta, Curr. Op. Struct. Biol., 2:593-596 (1992)), by substituting rodent CDRs or CDR sequences for the corresponding sequences of a human antibody. Accordingly, such humanized antibodies are chimeric antibodies (see, e.g., U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced, or exchanged so that the antigen binding site (variable region) is linked to a constant region of a different or altered class, effector function, and/or species, or an entirely different molecule which confers new properties to the chimeric antibody, e.g., an enzyme, toxin, hormone, growth factor, drug, etc.; or (b) the variable region, or a portion thereof, is altered, replaced, or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect, the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least about two, three, four, or more times the background, and more typically more than at least about 10 to about 100 times the background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow and Lane, Antibodies, A Laboratory Manual (1988) for a description of immunoassay formats and conditions that can be used to determine specific immunoreactivity).

The term “pharmaceutically acceptable salts” or “pharmaceutically acceptable carrier” is meant to include salts of the platinum-based compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides platinum-based compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain platinum-based compounds of the present invention can exist in unsolvated forms as well as solvated forms, including hydrated forms. In general, the solvated forms are equivalent to unsolvated forms and are intended to be encompassed within the scope of the present invention. Certain platinum-based compounds of the present invention may exist in multiple crystalline or amorphous forms. In general, all physical forms are equivalent for the uses contemplated by the present invention and are intended to be within the scope of the present invention.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a platinum-based compound described herein is administered at the same time, just prior to, or just after the administration of one or more additional cancer therapies such as chemotherapy, hormonal therapy, radiotherapy, or immunotherapy.

### III. Diagnostic and Prognostic Methods

In certain aspects, the present invention provides methods of diagnosing or providing a prognosis for a cancer therapy, e.g., therapy for a cancer that expresses at least one OCT marker (e.g., OCT1, OCT2 and/or OCT3) such as colorectal cancer or liver cancer. As used herein, the term “providing a prognosis” refers to providing a prediction of the probable course, recommended therapy, and outcome of a cancer or the likelihood of recovery from the cancer.

In certain instances, cancer patients with positive or high OCT expression have a longer disease-specific survival as compared to those with negative or low expression. As such, the level of OCT expression can be used as a prognostic indicator, with positive or high expression as an indication of a good prognosis, e.g., a longer disease-specific survival.

The methods of the present invention can also be useful for diagnosing the severity of a cancer, e.g., a cancer that expresses at least one OCT marker. As a non-limiting example, the level of OCT expression can be used to determine the stage or grade of a cancer such as colorectal cancer, e.g., according to the Tumor/Nodes/Metastases (TNM) system of classification (International Union Against Cancer, 6th edition, 2002), the Dukes staging system (Dukes, J. Pathol., 35:323 (1932)), or the Astler-Coller staging system (Astler et al., Ann. Surg., 139:846 (1954)). Typically, cancers are staged using a combination of physical examination, blood tests, and medical imaging. If tumor tissue is obtained via biopsy or surgery, examination of the tissue under a microscope can also provide pathologic staging. In certain instances, cancer patients with positive or high OCT expression have a more severe stage or grade of that type of cancer. As such, the level of OCT expression can be used as a diagnostic indicator of the severity of a cancer or of the risk of developing a more severe stage or grade of the cancer. In certain other instances, the stage or grade of a cancer assists a practitioner in determining the prognosis for the cancer and in selecting the appropriate cancer therapy.

Diagnosis or prognosis can involve determining the level of OCT expression (i.e., transcription or translation) in a patient and then comparing the level or localization to a baseline or range. Typically, the baseline value is representative of OCT expression levels in a healthy person not suffering from cancer. Variation of levels of a polypeptide or polynucleotide of the present invention from the baseline range (i.e., either up or down) indicates that the patient has a cancer or is at risk of developing a cancer. In some embodiments, the level of OCT expression is measured by taking a blood, urine, or tissue sample from a patient and measuring the amount of a polypeptide or polynucleotide of the present invention in the sample using any number of detection methods, such as those discussed herein.

Any antibody-based technique for determining a level of expression of a protein of interest can be used to measure the level of OCT expression in tumor tissue or cancerous cells. For example, immunoassays such as ELISA assays, immunoprecipitation assays, and immunohistochemical assays can be used to detect differential protein expression in patient samples. One skilled in the art will know of additional antibody-based techniques that can be used for determining a level of OCT expression according to the methods of the present invention. PCR assays can be used to detect expression levels of nucleic acids, as well as to discriminate between variants in genomic structure, such as insertion/deletion mutations, truncations, or splice variants.

In some embodiments, the expression of at least one OCT marker in a cancerous or potentially cancerous tissue may be evaluated by visualizing the presence and/or localization of the OCT marker in the subject. Any technique known in the art for visualizing tumors, tissues, or organs in live subjects can be used in the methods of the present invention. Preferably, the in vivo visualization of cancerous or potentially cancerous tissue is performed using an antibody that specifically binds to an OCT polypeptide (e.g., anti-OCT1 antibody, anti-OCT2 antibody, anti-OCT3 antibody). The visualization of cancerous or potentially cancerous tissue in live subjects can also be performed using any other molecule that specifically interacts or binds to an OCT transcript or to a polypeptide encoded by the transcript. Such agents may be used to visualize the patterns of gene expression and facilitate diagnosis or prognosis of cancers that express at least one OCT marker.

A detectable moiety can be coupled either directly or indirectly to anti-OCT antibodies using methods well known in the art. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

The detectable moiety can be visualized in live subjects using any device or method known in the art. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a detectable moiety linked to an anti-OCT antibody. Positron Emission Tomography (PET) is another suitable technique for detecting radiation in a subject to visualize tumors in living patients according to the methods of the present invention. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC, Santa Monica, Calif. Magnetic Resonance Imaging (MRI) or any other imaging technique known to one of skill in the art (e.g., radiography (i.e., X-rays), computed tomography (CT), fluoroscopy, etc.) is also suitable for detecting the radioactive emissions of radionuclides.

Various in vivo optical imaging techniques that are suitable for the visualization of fluorescent and/or enzymatic labels or markers include, but are not limited to, fluorescence microendoscopy (see, e.g., Flusberg et al., Optics Lett., 30:2272-2274 (2005)), fiber-optic fluorescence imaging (see, e.g., Flusberg et al., Nature Methods, 2:941-950 (2005)), fluorescence imaging using a flying-spot scanner (see, e.g., Ramanujam et al., IEEE Trans. Biomed. Eng., 48:1034-1041 (2001)), catheter-based imaging systems (see, e.g., Funovics et al., Radiology, 231:659-666 (2004)), near-infrared imaging systems (see, e.g., Mahmood et al., Radiology, 213:866-870 (1999)), fluorescence molecular tomography (see, e.g., Gurfinkel et al., Dis. Markers, 19:107-121 (2004)), and bioluminescent imaging (see, e.g., Dikmen et al., Turk. J. Med. Sci., 35:65-70 (2005)).

Anti-OCT antibodies, when conjugated to any of the above-described detectable moieties, can be administered in doses effective to achieve the desired image of tumor tissue or cancerous cells in a subject. Such doses may vary widely, depending upon the particular detectable label employed, the type of tumor tissue or cancerous cells subjected to the imaging procedure, the imaging equipment being used, and the like. However, regardless of the detectable moiety or imaging technique used, such detection is aimed at determining where one or more OCT markers are concentrated in a subject, with such concentration being an indicator of the location of a tumor or tumor cells. Alternatively, such detection is aimed at determining the extent of tumor regression in a subject, with the size of the tumor being an indicator of the efficacy of cancer therapy.

Diagnosis or prognosis can further involve determining the genotype of at least one OCT marker in a subject. For example, genotyping an OCT nucleic acid marker at a polymorphic site for alleles that result in decreased or increased organic cation transporter activity can be useful in diagnosing or providing a prognosis for cancers that express the OCT marker. In certain instances, an OCT nucleic acid marker that comprises a variant allele resulting in no or substantially reduced organic cation transporter activity is indicative of a cancer that does not express the OCT marker. A subject having this genotype would have a cancer that is resistant to platinum-based drug therapy and a poor prognosis for cancer using such therapy. Variant alleles in OCT1 and OCT2 which comprise polymorphisms suitable for detecting in the methods of the present invention are described in, e.g., Shu et al., Proc. Natl. Acad. Sci. U.S.A., 100:5902-5907 (2003); and Leabman et al., Pharmacogenetics, 12:395-405 (2002).

### IV. Assays

Any of a variety of assays, techniques, and kits known in the art can be used to determine the presence or level of an OCT protein or nucleic acid marker in a sample to determine an appropriate cancer therapy, diagnose or provide a prognosis for a cancer that expresses the OCT marker.

The methods of the present invention rely, in part, on determining whether or not OCT protein or nucleic acid (e.g., mRNA) is expressed in a sample. As used herein, the term “determining whether or not OCT protein (or nucleic acid) is expressed” refers to determining the presence or level of at least one OCT marker of interest (e.g., OCT1, OCT2, and/or OCT3) by using any quantitative or qualitative assay known to one of skill in the art. In certain instances, qualitative assays that determine the presence or absence of a particular trait, variable, or biochemical or serological substance (e.g., protein or antibody) are suitable for detecting each marker of interest. In certain other instances, quantitative assays that determine the presence or absence or the relative or absolute amount of RNA, protein, antibody, or activity are suitable for detecting each OCT marker of interest.

Typically, an OCT protein marker is analyzed using an immunoassay, although other methods are well known to those skilled in the art (e.g., the measurement of marker RNA levels). Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. The presence or amount of an OCT protein marker is generally determined using antibodies specific for the marker and detecting specific binding. For example, polyclonal antibodies directed to OCT1, OCT2, or OCT3 can be obtained from Alpha Diagnostics Intl. Inc. (San Antonio, Tex.).

Any suitable immunoassay can be utilized for determining whether or not OCT protein is expressed in a sample. A variety of immunoassay techniques, including competitive and non-competitive immunoassays, can be used (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to OCT protein can be detected directly or indirectly. Direct labels include fluorescent or luminescent tags, metals, dyes, radionuclides, and the like, attached to the antibody. An antibody labeled with iodine-125 (¹²⁵I) can be used for determining whether or not OCT protein is expressed in a sample. A chemiluminescence assay using a chemiluminescent antibody specific for an OCT protein marker is suitable for sensitive, non-radioactive detection of OCT protein levels. An antibody labeled with fluorochrome is also suitable for determining whether or not OCT protein is expressed in a sample. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of OCT marker levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Antigen capture assays can be useful in the methods of the present invention. For example, in an antigen capture assay, an antibody directed to an OCT protein marker is bound to a solid phase and sample is added such that OCT protein is bound by the antibody. After unbound proteins are removed by washing, the amount of bound marker can be quantitated using, for example, a radioimmunoassay (see, e.g., Harlow and Lane, Antibodies: A Laboratory Manual, Cold Spring Harbor Laboratory, New York, 1988)). Sandwich enzyme immunoassays can also be useful in the methods of the present invention. For example, in a two-antibody sandwich assay, a first antibody is bound to a solid support, and OCT protein is allowed to bind to the first antibody. The amount of OCT protein is quantitated by measuring the amount of a second antibody that binds the marker. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

Quantitative western blotting can also be used to detect or determine whether or not OCT protein is expressed in a sample. Western blots can be quantitated by well known methods such as scanning densitometry or phosphorimaging. In certain instances, autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art as described, e.g., in Parra et al., J. Vasc. Surg, 28:669-675 (1998).

Alternatively, a variety of immunohistochemistry (IHC) techniques can be used to determine whether or not OCT protein is expressed in a sample. As used herein, the term “immunohistochemistry” or “IHC” encompasses techniques that utilize the visual detection of fluorescent dyes or enzymes coupled (i.e., conjugated) to antibodies that react with the OCT protein marker using fluorescent microscopy or light microscopy and includes, without limitation, direct fluorescent antibody, indirect fluorescent antibody (IFA), anticomplement immunofluorescence, avidin-biotin immunofluorescence, and immunoperoxidase assays. An IFA assay, for example, is useful for determining whether a sample is positive for OCT protein, the level of that marker, and/or the staining pattern of that marker. The concentration of OCT protein in a sample can be quantitated, e.g., through endpoint titration or through measuring the visual intensity of fluorescence compared to a known reference standard.

The presence or level of an OCT protein marker can also be determined by detecting or quantifying the amount of the purified marker. Purification of OCT protein can be achieved, for example, by high pressure liquid chromatography (HPLC), alone or in combination with mass spectrometry (e.g., MALDI/MS, MALDI-TOF/MS, tandem MS, etc.). Qualitative or quantitative detection of OCT protein can also be determined by well-known methods including, without limitation, Bradford assays, Coomassie blue staining, silver staining, assays for radiolabeled protein, and mass spectrometry.

The analysis of a plurality of OCT protein markers may be carried out separately or simultaneously with one test sample. For separate or sequential assay of OCT protein markers, suitable apparatuses include clinical laboratory analyzers such as the ElecSys (Roche), the AxSym (Abbott), the Access (Beckman), the ADVIA®, the CENTAUR® (Bayer), and the NICHOLS ADVANTAGE® (Nichols Institute) immunoassay systems. Preferred apparatuses or protein chips perform simultaneous assays of a plurality of OCT protein markers on a single surface. Particularly useful physical formats comprise surfaces having a plurality of discrete, addressable locations for the detection of a plurality of different biomarkers. Such formats include protein microarrays, or “protein chips” (see, e.g., Ng et al., J. Cell Mol. Med., 6:329-340 (2002)) and certain capillary devices (see, e.g., U.S. Pat. No. 6,019,944). In these embodiments, each discrete surface location may comprise antibodies to immobilize one or more OCT protein markers for detection at each location. Surfaces may alternatively comprise one or more discrete particles (e.g., microparticles or nanoparticles) immobilized at discrete locations of a surface, where the microparticles comprise antibodies to immobilize one or more OCT protein markers for detection.

In addition to the above-described assays for determining whether or not OCT protein is expressed in a sample, analysis of OCT marker mRNA levels using routine techniques such as Northern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. The mRNA expression of a gene of interest is typically evaluated in vitro on a sample collected from the subject in comparison to a normal or reference sample. Applicable PCR amplification techniques are described in, e.g., Ausubel et al., Theophilus et al., and Innis et al., supra. General nucleic acid hybridization methods are described in Anderson, “Nucleic Acid Hybridization,” BIOS Scientific Publishers, 1999. Amplification or hybridization of a plurality of transcribed nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of the genotype of an OCT nucleic acid marker can be performed using techniques known in the art including, without limitation, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell. Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), and sequencing by hybridization (Chee et al., Science, 274:610-614 (1996); Drmanac et al., Science, 260:1649-1652 (1993); Drmanac et al., Nature Biotech., 16:54-58 (1998)). Non-limiting examples of electrophoretic analysis include slab gel electrophoresis such as agarose or polyacrylamide gel electrophoresis, capillary electrophoresis, and denaturing gradient gel electrophoresis. Other methods for genotyping a subject at a polymorphic site in an OCT nucleic acid marker include, e.g., the INVADER® assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis.

Analysis of whether or not an OCT nucleic acid has been gene amplified or deleted in a sample can also be used in the pharmacogenetic, diagnostic and prognostic methods of the present invention. Any method known in the art for detecting or determining the presence or level of gene amplification or deletion of one or more of the OCT nucleic acid markers described herein is suitable for use in the present invention. In some embodiments, the presence or level of gene amplification or deletion of one or more OCT nucleic acid markers can be determined by DNA-based techniques such as PCR or Southern blot analysis or by molecular cytogenetic techniques such as fluorescence in situ hybridization (FISH), chromogenic in situ hybridization (CISH), and immunohistochemistry. Other techniques include genome-wide scanning of amplified chromosomal regions with comparative genomic hybridization for the detection of amplified regions in tumor DNA (see, e.g., Kallioniemi et al., Science, 258:818-821 (1992)) and the detection of gene amplification by genomic hybridization to cDNA microarrays (see, e.g., Heiskanen et al., Cancer Res., 60:799-802 (2000)). One skilled in the art will know of additional gene amplification or deletion techniques that can be used to detect or determine a level of an amplified gene that corresponds to one or more OCT nucleic acid markers of the present invention.

The analysis of OCT protein or nucleic acid markers can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

### V. Selection of Antibodies

The generation and selection of antibodies not already commercially available for detecting or determining the levels of OCT protein markers may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol., Vol. 182, 1990; Solid Phase Peptide Synthesis, Greg B. Fields, ed., Meth. Enzymol., Vol. 289, 1997; Kiso et al., Chem. Pharm. Bull., 38:1192-99 (1990); Mostafavi et al., Biomed. Pept. Proteins Nucleic Acids, 1:255-60, (1995); Fujiwara et al., Chem. Pharm. Bull., 44:1326-31 (1996). The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, eds., Cold Spring Harbor Laboratory, Cold Spring Harbor, N.Y., 1988. One skilled in the art will also appreciate that binding fragments or Fab fragments which mimic antibodies can also be prepared from genetic information by various procedures (see, e.g., Antibody Engineering: A Practical Approach, Borrebaeck, ed., 1995, Oxford University Press, Oxford; J. Immunol., 149:3914-3920 (1992)).

In addition, numerous publications have reported the use of phage display technology to produce and screen libraries of polypeptides for binding to a selected target (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means (see, e.g., U.S. Pat. No. 6,057,098).

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or group of antibodies is then placed into the respective microtiter wells and incubated for about 30 min to 2 h. The microtiter wells are then washed and a labeled secondary antibody (e.g., an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ, certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the present invention.

### VI. Gene Therapy

The present invention also provides methods of treating or inhibiting a cancer such as a therapy resistant cancer or a cancer that does not express OCT markers by administering a therapeutically effective amount of a nucleic acid encoding OCT1, OCT2, and/or OCT3, e.g., for gene therapy. As used herein, the term “gene therapy” refers to a therapeutic approach for introducing a specific polynucleotide into cells (e.g., cancer cells) to restore missing or abnormal gene expression; to increase reduced gene expression; to provide expression of a gene not typically expressed in the cells; or to inhibit gene expression. Examples of suitable gene therapy techniques include, without limitation, introducing wild-type copies of a gene into cancer cells that are missing expression of the gene or that have abnormal expression of the gene, inhibiting the expression of genes such as oncogenes in cancer cells, introducing genes into cancer cells that make them more vulnerable to cytotoxic therapy (e.g., chemotherapy, radiotherapy, immunotherapy, hormonal therapy, etc.), introducing genes into cancer cells that make them more easily detected and destroyed by the body's immune system, and inhibiting genes in cancer cells that are involved in angiogenesis.

In preferred embodiments of the present invention, the methods of treating or inhibiting a cancer involve administering a therapeutically effective amount of one or more OCT nucleic acids that increase OCT expression to make cancer cells more sensitive to cytotoxic therapy with platinum-based drugs. Without being bound to any particular theory, the introduction of one or more OCT nucleic acids into cancer cells potentiates the effect of other cancer therapies by sensitizing the cells to such cytotoxic therapies. As a result, therapy resistant cancers can be effectively treated with gene therapy using OCT nucleic acids.

A variety of techniques are available for delivering the nucleic acid into cells for gene therapy including, but not limited to, in vivo and ex vivo techniques. For example, in vivo techniques can rely on the use of a virus (e.g., adenovirus) containing the desired nucleic acid sequence to be introduced into cancer cells. Alternatively, in vivo techniques can rely on the use of delivery systems that are complexed with or encapsulate the nucleic acid, e.g., lipoplexes or liposomal delivery systems. One skilled in the art will also appreciate that the nucleic acid can be administered as a naked molecule, e.g., injected directly into the tumor. Ex vivo techniques involve removing cells from a patient, introducing the desired nucleic acid sequence into the cells, and placing the cells back into the patient. Suitable cells include cancer cells as well as cells of the immune system (e.g., to stimulate an immune response to the cancer cells). For example, cancer cells that have been removed and genetically altered can be injected back into the patient in hopes that immune cells will destroy them and any other cancer cells that resemble them. This approach may be useful in making the cancer cells more visible to the immune system, which often has a difficult time finding and attacking cancer cells in the body. Cells of the immune system such as dendritic cells can also be removed and genetically altered to make them more likely to attack cancer cells once they are put back into the body.

Numerous techniques are known in the art for the introduction of foreign genes into cells and may be used to construct the recombinant cells for purposes of gene therapy. Techniques which may be used include, but are not limited to, cell fusion, chromosome-mediated gene transfer, micro cell-mediated gene transfer, transfection, transformation, transduction, electroporation, infection (e.g., recombinant DNA viruses, recombinant RNA viruses), spheroplast fusion, microinjection, DEAE dextran, calcium phosphate precipitation, liposomes, lysosome fusion, synthetic cationic lipids, use of a gene gun or a DNA vector transporter, etc. For various techniques for transformation or transfection of mammalian cells, see, e.g., Keown et al., Methods Enzymol. 185:527-37 (1990); Sambrook et al., Molecular Cloning, A Laboratory Manual, Third Edition, Cold Spring Harbor Laboratory Press, N.Y. (2001).

### VII. Methods of Administration and Pharmaceutical Compositions

As described herein, molecules and compounds such as platinum-based drugs that have been identified as substrates for one or more OCTs are useful in treating cancers that express OCT protein or nucleic acid. For therapeutic applications, the platinum-based drugs of the present invention can be administered alone or co-administered in combination with conventional chemotherapy, radiotherapy, hormonal therapy, and/or immunotherapy.

As a non-limiting example, the platinum-based drugs described herein can be co-administered with conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), and the like.

The platinum-based drugs described herein can also be co-administered with conventional hormonal therapaeutic agents including, but not limited to, steroids (e.g., dexamethasone), finasteride, aromatase inhibitors, tamoxifen, and gonadotropin-releasing hormone agonists (GnRH) such as goserelin.

Additionally, the platinum-based drugs described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the platinum-based drugs described herein can be co-administered with conventional radiotherapeutic agents including, but not limited to, radionuclides such as ⁴⁷sc, ⁶⁴CU, ⁶⁷CU, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In some embodiments, the compositions of the present invention comprise the platinum-based drugs described herein and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid, solid, or semi-solid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Examples of solid or semi-solid carriers include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, 17^{th} ed., 1989).

The pharmaceutical compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate.

Formulations suitable for oral administration can comprise: (a) liquid solutions, such as an effective amount of a packaged platinum-based drug suspended in diluents, e.g., water, saline, or PEG 400; (b) capsules, sachets, or tablets, each containing a predetermined amount of a platinum-based drug, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a platinum-based drug in a flavor, e.g., sucrose, as well as pastilles comprising the polypeptide or peptide fragment in an inert base, such as gelatin and glycerin or sucrose and acacia emulsions, gels, and the like, containing, in addition to the polypeptide or peptide, carriers known in the art.

The platinum-based drug of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which comprises an effective amount of a packaged platinum-based drug with a suppository base. Suitable suppository bases include natural or synthetic triglycerides or paraffin hydrocarbons. In addition, it is also possible to use gelatin rectal capsules which contain a combination of the platinum-based drug of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a platinum-based drug. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In therapeutic use for the treatment of cancer, the platinum-based drug utilized in the pharmaceutical compositions of the present invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the platinum-based drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular platinum-based drug in a particular patient. Determination of the proper dosage for a particular situation is within the skill of the practitioner. Generally, treatment is initiated with smaller dosages which are less than the optimum dose of the platinum-based drug. Thereafter, the dosage is increased by small increments until the optimum effect under circumstances is reached. For convenience, the total daily dosage may be divided and administered in portions during the day, if desired.

### VIII. Compositions, Kits, and Integrated Systems

The present invention provides compositions, kits, and integrated systems for practicing the assays described herein using the polypeptides or polynucleotides described herein, antibodies specific for the polypeptides or polynucleotides described herein, etc.

In one embodiment, the present invention provides assay compositions for use in solid phase assays. Such compositions can include, for example, one or more polypeptides or polynucleotides of the present invention immobilized on a solid support, and a labeling reagent. In each case, the assay compositions can also include additional reagents that are desirable for hybridization. Modulators of expression or activity of the polypeptides or polynucleotides of the present invention can also be included in the assay compositions.

In another embodiment, the present invention provides kits for carrying out the therapeutic, diagnostic, and prognostic assays described herein. The kits typically include one or more probes that comprise an antibody or nucleic acid sequence that specifically binds to the polypeptides or polynucleotides of the present invention, and a label for detecting the presence of the probe. The kits can find use, for example, for measuring the levels of OCT protein or OCT transcripts, or for measuring OCT activity to a target substrate. The kits may also include several polynucleotide sequences encoding polypeptides of the present invention. Kits can include any of the compositions noted above, and optionally further include additional components such as instructions to practice a high-throughput method of assaying for an effect on expression of the genes encoding the polypeptides of the present invention, or on activity of the polypeptides of the present invention, one or more containers or compartments (e.g., to hold the probe, labels, or the like), a control modulator of the expression or activity of polypeptides of the present invention, a robotic armature for mixing kit components, or the like.

In yet another embodiment, the present invention provides integrated systems for high-throughput screening of potential modulators for an effect on the expression or activity of the polypeptides of the present invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety. A number of robotic fluid transfer systems are available or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments described herein, e.g., by digitizing the image and storing and analyzing the image on a computer. A variety of commercially available peripheral equipment and software is available for digitizing, storing, and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the present invention are easily used for viewing any sample, e.g., by fluorescent or dark field microscopic techniques.

### IX. Examples

The following example is offered to illustrate, but not to limit, the claimed invention.

### Example 1

### Organic Cation Transporters are Determinants of Oxaliplatin Cytotoxicity

This example characterizes the interaction of cisplatin, carboplatin, and oxaliplatin with human OCT1, OCT2, and OCT3. In particular, this example provides a determination of whether OCTs play a role in the cytotoxicity of these and related platinum-based drugs, a determination of whether interactions with OCTs contribute to the differential antitumor specificity of oxaliplatin versus cisplatin, and an understanding of the underlying chemical principles that determine these differences. The present study demonstrates for the first time that OCT1 and OCT2 play an important role in mediating the uptake and consequent cytotoxicity of oxaliplatin, but not cisplatin or carboplatin. Structure-activity relationship studies indicate that the 1,2-diaminocyclohexane (DACH) moiety of oxaliplatin is an important pharmacophore for its interaction with OCTs and that an organic component on the non-leaving portion of the platinum complexes is essential. Finally, the present study indicates that interactions with OCT1 and OCT2 are likely to be important contributors to the sensitivity of cancers such as colorectal cancer to oxaliplatin.

### Summary

Although the platinum-based anticancer drugs cisplatin, carboplatin, and oxaliplatin have similar DNA-binding properties, only oxaliplatin is active against colorectal tumors. The present study illustrates that human OCT1 and OCT2 markedly increase oxaliplatin, but not cisplatin or carboplatin, accumulation and cytotoxicity in OCT-transfected cells. The cytotoxicity of oxaliplatin was greater than that of cisplatin in six colon cancer cell lines, but decreased by the OCT inhibitor, cimetidine, to a level similar to that of cisplatin. Structure-activity studies identified the requirement for organic functionalities on non-leaving groups coordinated to platinum as being important for selective uptake by OCTs. These results indicate that OCTs are major determinants of oxaliplatin cytotoxicity and contribute to its antitumor specificity for colorectal cancer.

### Materials and Methods

### Drugs and Reagents

Cisplatin, carboplatin, oxaliplatin, cimetidine, disopyramide, N-methyl-4-phenylpyridinium (MPP⁺), and thiazolyl blue tetrazolium bromide were purchased from Sigma (St. Louis, Mo.). Solutions of carboplatin (10 mM) and oxaliplatin (5 mM) were freshly prepared in water. A solution of cisplatin (2 mM) was made in 1× phosphate buffered saline (PBS). These stock solutions were immediately aliquoted and stored frozen at −20° C. and discarded one month after preparation. [Methyl-3H]-MPP⁺ was purchased from Perkin Elmer (Boston, Mass.) and tetraethylammonium (TEA) bromide [ethyl-1-¹⁴C] was purchased from American Radiolabeled Chemicals (St. Louis, Mo.). Hygromycin B and G418 were purchased from Invitrogen (Carlsbad, Calif.). The cell culture medium Dubecco's Modified Eagle Medium (DMEM), RPMI, and fetal bovine serum (FBS) were obtained from the Cell Culture Facility of the University of California, San Francisco (San Francisco, Calif.).

### Cell Lines and Transfection

Madin-Darby canine kidney (MDCK) cells stably transfected with the full length human OCT1 cDNA (MDCK-hOCT1) and with the empty vector (MDCK-MOCK) were established as described in Shu et al., Proc. Natl. Acad. Sci. U.S.A., 100:5902-5907 (2003). Human embryonic kidney (HEK) 293 cells transfected with pcDNA5/FRT vector (Invitrogen) containing the full length human OCT2 cDNA (HEK-hOCT2) and with the empty vector (HEK-MOCK) were established using Lipofectamine™ 2000 (Invitrogen) per manufacturer's instructions. The stable clones were selected with 75 μg/ml of hygromycin B. HEK 293 cells transfected with pcDNA3 vector containing the full length human OCT3 cDNA (HEK-hOCT3) and with the empty vector (HEK-MOCK) were also established using Lipofectamine™ 2000. The stable clones were selected with 600 μg/ml G418. The pcDNA3 vector containing the full length human OCT3 cDNA was obtained from Dr. Bonisch (Institute of Pharmacology and Toxicology, University of Bonn, Germany). All the colon cancer cell lines (LS180, SW620, DLD, HCT116, HT20, and RKO) used in the present study were obtained from American Type Culture Collection (Manassas, Va.).

### Cell Culture

The culture medium for stably transfected MDCK and HEK 293 cells was DMEM supplemented with 10% FBS and 100 units/ml penicillin and 100 μg/ml streptomycin (Invitrogen). To maintain transgene expression, G418 (Invitrogen, 600 μg/ml) was added to the culture medium for MDCK transfected cells, human OCT3 transfected HEK 293 cells (HEK-hOCT3), and the corresponding HEK-MOCK cells. Hygromycin B (Invitrogen, 75 μg/ml) was added to the culture medium for human OCT2 transfected HEK 293 cells (HEK-hOCT2) and the corresponding HEK-MOCK cells. The culture medium for all the colon cancer cell lines (RKO, DLD, HT29, HCT116, LS180, and SW620) was RPMI containing 10% FBS, 100 units/ml penicillin and 100 μg/ml streptomycin. All the cells were grown at 37° C. in a humidified atmosphere with 5% CO₂/95% air.

### Drug Sensitivity Assay

Cytotoxicity of the platinum-based compounds was measured by MTT (thiazolyl blue tetrazolium bromide) assay. Cells were seeded in 100 μl of the culture medium without any antibiotics in 96-well plates at a predetermined cell density. For HEK 293 cells, poly-D-lysine coated plates were used. After overnight incubation, the platinum-based compounds were then added to the culture medium to give the indicated final concentrations. For the OCT inhibitor studies, the inhibitors (i.e., disopyramide or cimetidine) were added to the medium at a specified concentration immediately before the addition of the platinum-based compounds. After incubation for a specified time period, the drug-containing medium was replaced with fresh, drug-free medium and the incubation was continued for a total of 72 hours (start from the addition of platinum-based compounds). Then, the MTT assay was performed similarly as described in Alley et al., Cancer Res., 48:589-601 (1988). The IC₅₀ (i.e., the drug concentration which inhibits 50% of cell growth) values were obtained by fitting the percent of the maximal cell growth at different drug concentrations (F) with the equation, F=100×(1−C^{γ}/(IC₅₀^{γ}+C^{γ})), using WinNonlin (Pharsight, Mountain View, Calif.). The maximal cell growth was the cell growth in the medium without any platinum-based compounds; C is the concentration of the platinum-based compound, and γ is the slope factor.

### Cellular Uptake of TEA or MPP⁺

MDCK or HEK 293 cells were grown in 24-well plates to >90% confluence in the culture medium without any antibiotics. The poly-D-lysine coated plates were used for HEK 293 cells. The cells were washed with 1×PBS first and then incubated in the uptake buffer (1×PBS) containing 10 μM ¹⁴C-TEA or 2 μM ³H-MPP⁺ as specified. For the OCT inhibitor studies, the indicated OCT inhibitor (i.e., disopyramide or cimetidine) was added to the uptake buffer at specified concentration together with the radioactive substrate. The uptake was performed at room temperature for 2 min (¹⁴C-TEA uptake) or 5 min (3H-MPP⁺) and then the cells were washed with ice-cold PBS for three times. The cells were then lysed with the lysis buffer (0.1 N NaOH, 0.1% SDS) for scintillation counting and BCA protein assay (Pierce, Rockford, Ill.) to determine the uptake.

### Cellular Accumulation of Platinum

The cellular accumulation of platinum was determined as described in Holzer et al., Mol. Pharmacol., 66:817-823 (2004) with some modifications. Briefly, the cells were grown in 100 mm×20 mm dishes in the culture medium without any antibiotics to over 90% confluence. For HEK 293 cells, poly-D-lysine coated dishes were used. For platinum accumulation, the cells were incubated in the culture medium containing the indicated concentrations of the platinum compounds at 37° C. in 5% CO₂ for 2 hr unless specified. After incubation, the dishes were immediately placed on ice and the cells were washed with 6 ml of ice-cold PBS for three times and collected with a rubber policeman. The cell pellets were obtained by centrifugation at 400×g and at 4° C. for 15 min. For the OCT inhibitor studies, the incubation medium also contained the indicated inhibitor (i.e., disopyramide or cimetidine) in addition to the platinum-based compounds. The resulting cell pellets were then dissolved into 200 μl of 70% nitric acid at 65° C. for at least 2.5 hours, and then distilled water containing 10 ppb of iridium (Sigma) and 0.1% Triton X-100 was added to the samples to dilute nitric acid to 7%. The platinum content was measured by inductively plasma coupled mass spectrometry (ICP-MS) in the Analytical Facility at the University of California at Santa Cruz (Santa Cruz, Calif.). Cell lysates from a set of identical cultures were used for BCA protein assay.

### Platinum-DNA Adduct Formation

The platinum content associated with genomic DNA was determined as described in Samimi et al., Clin. Cancer Res., 10:4661-4669 (2004) with some modifications. Briefly, the cells were grown in 100 mm×20 mm dishes in the culture medium without any antibiotics to over 90% confluence. For HEK 293 cells, poly-D-lysine coated dishes were used. Then, the cells were incubated in the culture medium containing the specified concentrations of the platinum-based compounds at 37° C. in 5% CO₂ for 2 hours (or 25 min as specified). In some experiments, phosphate buffer (PB: 1.06 mM KH₂PO₄, 2.97 mM Na₂HPO₄, pH 7.4) containing 155 mM NaCl (PB—Cl buffer) or 103 mM Na₂SO₄ (PB—SO₄ buffer) was used instead of the culture medium as specified. For the OCT inhibitor study, the incubation medium (buffer) also contained disopyramide or cimetidine). If PB—Cl or PB—SO₄ buffer was used, the cells were washed with the same buffer once before drug incubation. After incubation, the cells were washed with ice-cold PBS, scraped, and pelleted. Genomic DNA was isolated from the cell pellets using the Wizard® Genomic DNA Purification Kit (Promega, Madison, Wis.) following the manufacturer's instructions. Briefly, the cells were lysed with Nuclei Lysis Solution. After RNA digestion and protein precipitation, the lysates were centrifuged and the resulting supernatant was aliquoted. The genomic DNA prepared from two different aliquots of the supernatant was used for platinum and DNA content determination, respectively. For the determination of platinum, the DNA samples were treated with 70% nitric acid at 65° C. and diluted in the same way as described above. The platinum content was analyzed using ICP-MS and the DNA content was measured by absorption spectroscopy.

### RNA Isolation

Cultured cells were grown in 100 mm×20 mm dishes to 70-80% confluence. Total RNA was isolated using RNeasy® Mini Kit (Qiagen, Valencia, Calif.) following the manufacturer's instructions, quantified by spectroscopy, and stored at −80° C. until use. Samples of tumor and normal colon mucosa were collected from colon cancer resection from Department of Surgery, Queen Mary Hospital, University of Hong Kong. Tissues were frozen in liquid nitrogen within half an hour after they were resected. Normeoplastic mucosa from colon was dissected free of muscle and histologically confirmed to be tumor free by frozen section. Total RNA was extracted using Trizol (Invitrogen). This study was approved by the Ethics Committee of the University of Hong Kong and the Internal Review Board of the University of California, San Francisco.

### RT-PCR

The first-strand cDNA was synthesized from 2 μg of total RNA using SuperScript™ III First-Strand Synthesis System for RT-PCR kit (Invitrogen) in a 20 μl reaction mixture, and the random hexamers were used as the primer. The sense and antisense primers for human OCT1 were 5′-CTG TGT AGA CCC CCT GGC TA-3′ (SEQ ID NO:1) and 5′-GTG TAG CCA GCC ATC CAG TT-3′ (SEQ ID NO:2), corresponding to the nucleotide positions 408-427 and 751-770 (Accession No. NM_{—}003057), respectively, and the size of the expected PCR product was 363 bp. The sense and antisense primers for human OCT2 were 5′-CCT GGT ATG TGC CAA CTC CT-3′ (SEQ ID NO:3) and 5′-CAC CAG GAG CCC AAC TGT AT-3′ (SEQ ID NO:4), corresponding to the nucleotide positions 590-609 and 904-923 (Accession No. NM_{—}003058), respectively, and the size of the expected PCR product was 334 bp. The sense and antisense primers for human OCT3 were 5′-ATC GTC AGC GAG TTT GAC CT-3′ (SEQ ID NO:5) and 5′-TTG AAT CAC GAT TCC CAC AA-3′ (SEQ ID NO:6), corresponding to the nucleotide positions 445-464 and 749-768 (Accession No. NM_{—}021977), respectively, and the size of the expected PCR product was 324 bp. The sense and antisense primers for human GAPDH were 5′-AAT CCC ATC ACC ATC TTC CA-3′ (SEQ ID NO:7) and 5′-TGT GGT CAT GAG TCC TTC CA-3′ (SEQ ID NO:8), corresponding to the nucleotide positions 289-308 and 587-606 (Accession No. NM_{—}002046), respectively, and the size of the expected PCR product was 318 bp. The sense and antisense primers for dog GAPDH were 5′-GGT GAT GCT GGT GAG TA-3′ (SEQ ID NO:9) and 5′-GTG GAA GCA GGG ATG ATG TT-3′ (SEQ ID NO:10), corresponding to the nucleotide positions 256-275 and 607-626 (Accession No. AB038240), respectively, and the size of the expected PCR product was 371 bp. All sets of primers were designed to anneal with sequences in different exons of the genes. An annealing temperature of 58° C. was used for PCR amplification. A cycle number of 40 was used for the detection of human OCT1 and OCT2 in the colon cancer cell lines and colon tissue samples. A cycle of 30 was used to detect human OCT1, OCT2, or OCT3 in the corresponding OCT-transfected cells and the MOCK cells. For the detection of human or dog GAPDH, a PCR cycle number of 30 was used in all the conditions.

### Synthesis of Platinum Analogs

Potassium tetrachloroplatinate(II) was obtained from Engelhard Corp. and the starting materials cisplatin and potassium amminetrichloroplatinate(II) were synthesized as described in Dhara, Indian Journal of Chemistry, 8:193-194 (1970) and Giandomenico, Inorganic Chemistry, 34:1015-1021 (1995). ¹H NMR spectra were acquired on a Varian 300 MHz spectrometer. FT-IR spectra were measured on an Avatar 380 FT-IR (Thermo Nicolet, Waltham, Mass.). ESI-MS spectra were obtained on an Agilent Technologies 1100 Series LCMS instrument. Previously described procedures were used to prepare [Pt(Cl₂)(en)] (Dhara, supra), cis-[Pt(NH₃)(cy)Cl₂] (Giandomenico, supra), and [Pt(R,R-DACH)Cl₂] (Hoeschele, Inorganic Chemistry, 27:4106-4113 (1988)). The [Pt(S,S-DACH)Cl₂] and [Pt(S,S-DACH)oxalato] compounds were synthesized as described in Kidani, J. Medicinal Chemistry, 21:1315-1318 (1978). FTIR and ¹H NMR spectra of all compounds matched literature spectra.

Preparation of [Pt(NH₃)₂(trans-1,2-(OCO)₂C₆H₁₀)]: This compound was prepared as described for the Pt-DACH derivative (Al-Allaf, Transition Metal Chemistry, 28:717-721 (2003)). Solubility problems, similar to those reported for the DACH compound, prevented analysis by NMR spectroscopy. IR (KBr, cm⁻¹) 3266 (sh), 2920 (s), 2850 (s), 1618 (s), 1556 (sh), 1384 (vs), 1279 (w), 1222 (m), 1111 (w), 1030 (w), 772 (w), 719 (w), 588 (b). ESI-MS: [M+H]⁺=400.2 amu (observed), 400.3 amu (calculated).

Preparation of [Pt(R,R-DACH)(H₂O)₂]²⁺: [Pt(R,R-DACH)Cl₂] was dissolved in distilled water (200 μM) and incubated with silver nitrate (400 μM) in the dark for 10 hours. [Pt(R,R-DACH)(H₂O)₂]²⁺ was obtained by filtering the reaction mixture to remove the silver chloride precipitate.

### Statistical Analysis

The differences between the mean values were analyzed for significance using Student's t test. P values<0.05 were considered statistically significant.

### Results

### OCT Expression and Function in Stably Transfected Cell Lines

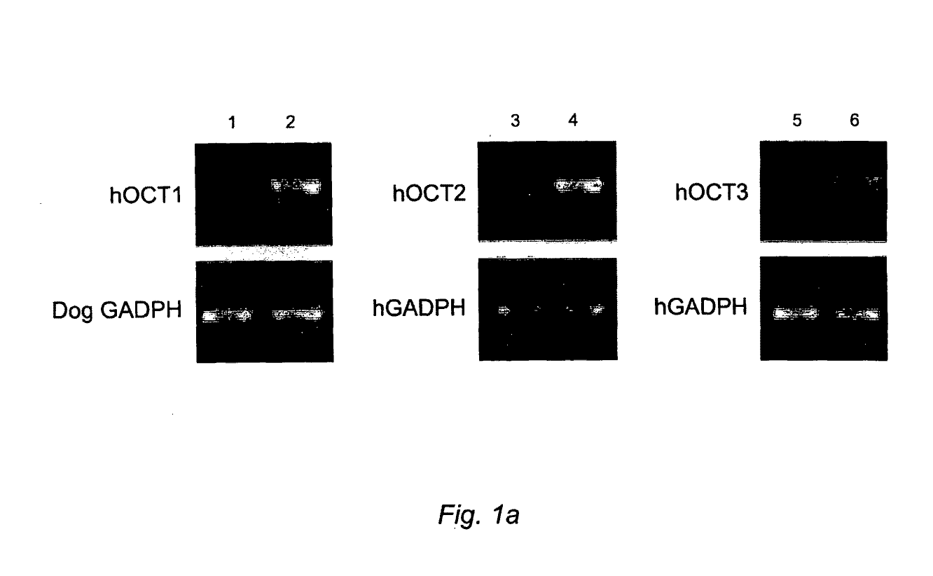

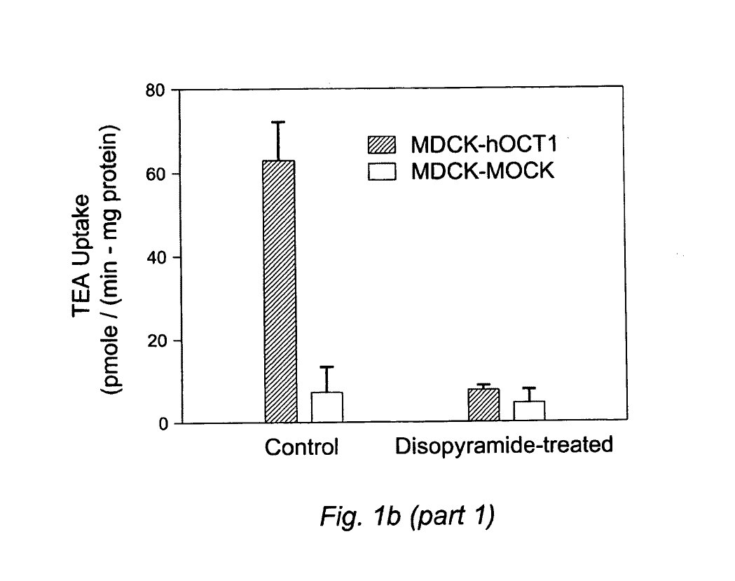

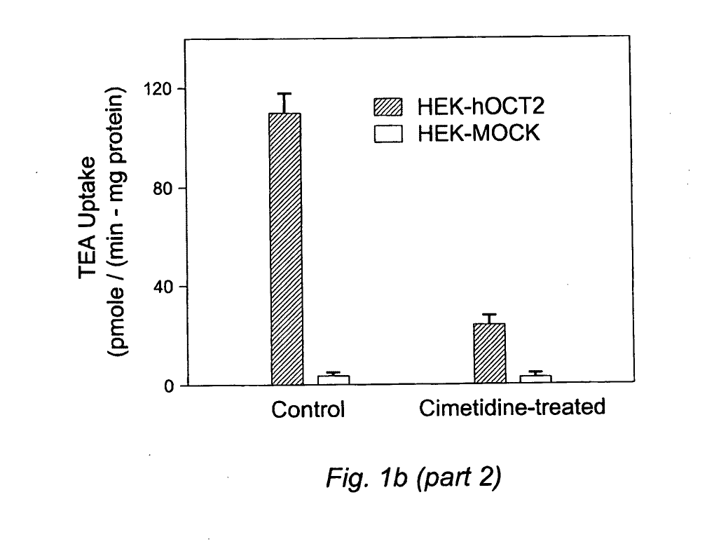

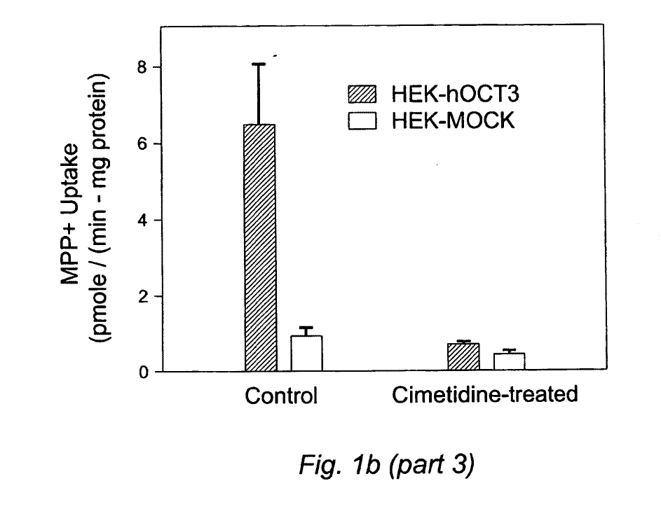

The expression and function of human OCTs in stably transfected cells was confirmed by RT-PCR and by examining the uptake of model OCT substrates (i.e., TEA for OCT1 and OCT2; MPP⁺ for OCT3). The expression of the mRNA transcripts of OCT1, OCT2, and OCT3 and uptake of model compounds were clearly much higher in OCT-transfected cells (MDCK-hOCT1, HEK-hOCT2, or HEK-hOCT3) in comparison to empty vector-transfected control counterparts (MOCK cells) (FIGS. 1A and 1B). OCT inhibitors (disopyramide (120 μM) for OCT1, cimetidine (1.5 mM) for OCT2 and OCT3) substantially decreased the uptake of the model compounds in the OCT-transfected cells (p<0.001) (FIG. 1B).

### Effect of OCTs on the Cytotoxicity of Cisplatin, Carboplatin, and Oxaliplatin

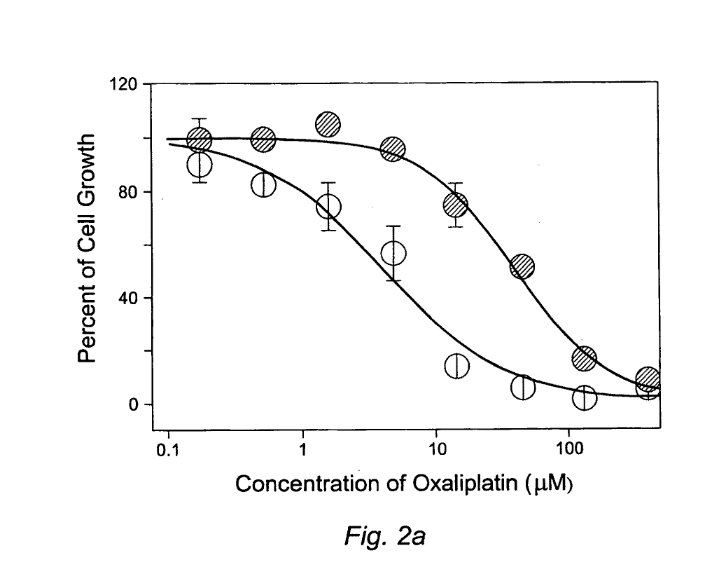

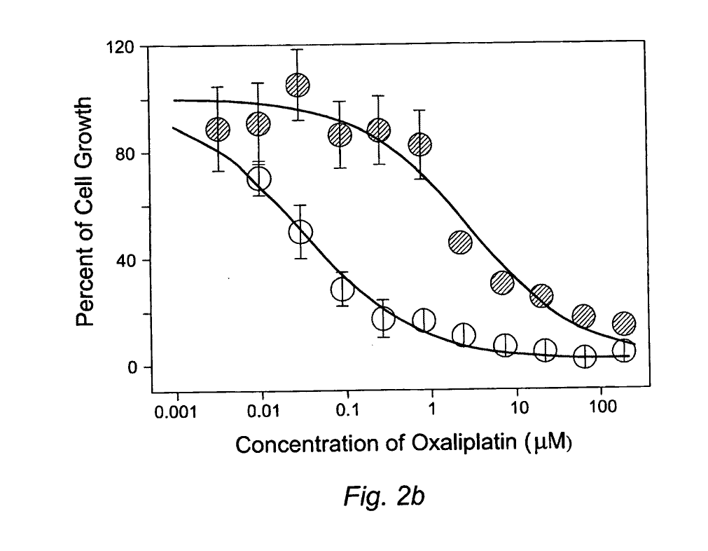

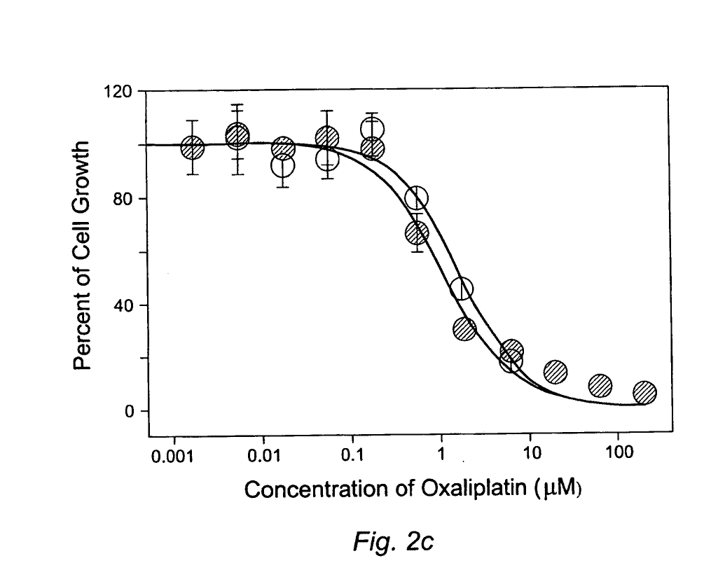

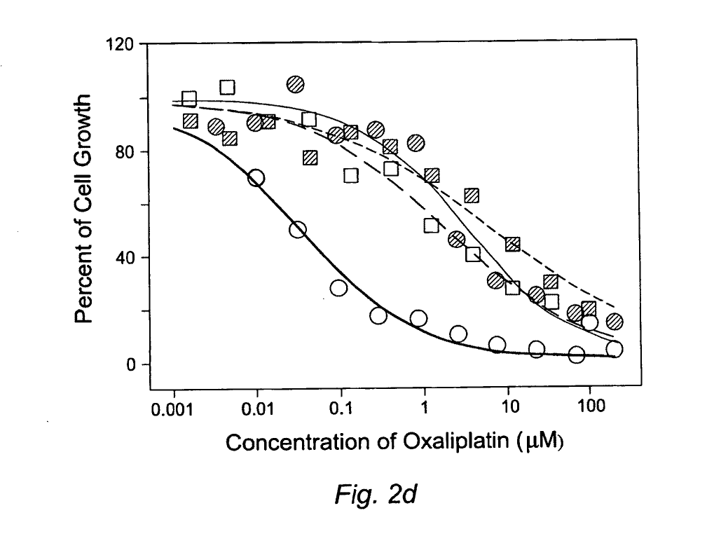

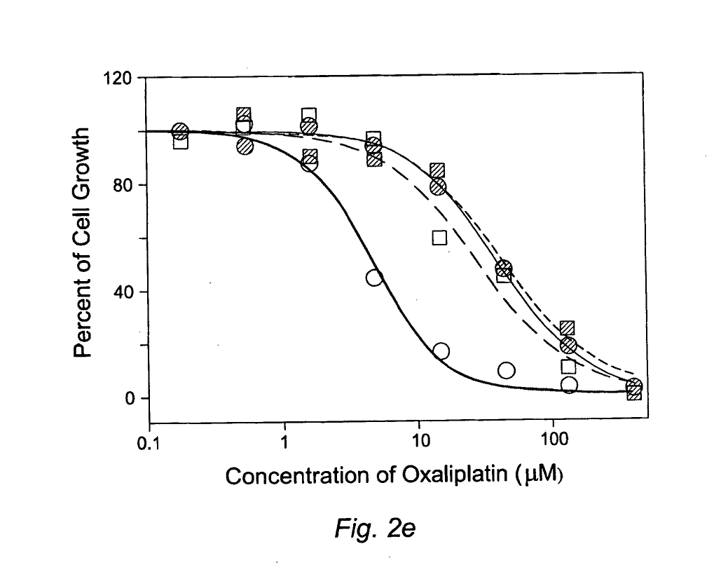

The IC₅₀ values of oxaliplatin, determined in MTT assays, in MDCK-MOCK cells after different time periods (7, 24, and 72 hr) of drug exposure were all significantly higher than those in MDCK-hOCT1 cells. As shown in Table 1 and FIG. 2A, the resistance factor (RF), defined as the ratio of the IC₅₀ value in MOCK cells to that in the corresponding OCT-transfected cells, ranged from 5.73 to 8.48 (p<0.01 to p<0.001). In contrast, the IC₅₀ values of both cisplatin and carboplatin were similar in the OCT1-transfected and in the MDCK-MOCK cells with RF values close to unity (p>0.05) (Table 1). Co-incubation with a known OCT1 inhibitor, disopyramide (150 μM), substantially increased the IC₅₀ value of oxaliplatin in MDCK-hOCT1 (control vs. disopyramide-treated: 3.79±1.57 μM vs. 22.8±10.5 μM) by 6.01-fold (p<0.05) with little effect in MDCK-MOCK (control vs. disopyramide-treated: 30.4±9.28 vs. 32.2±13.0 μM, p>0.05) tested in parallel (FIG. 2D). Disopyramide itself did not manifest any cytotoxicity up to a concentration of 400 μM under the same test conditions. These results indicate that OCT1 enhances the cytotoxicity of oxaliplatin, but not that of cisplatin or carboplatin.

TABLE 1
Drug sensitivity of cisplatin, carboplatin, and oxaliplatin
in OCT-transfected cells: Cytotoxicity, expressed as IC₅₀, of
the platinum-based drugs in MDCK-MOCK and MDCK-hOCT1 cells.

| Platinum | Drug Exposure | MDCK-MOCK | MDCK-hOCT1 | |
|---|---|---|---|---|
| Drugs | Time (hour) | (μM) | (μM) | RF |
| cisplatin | 7 | 19.6 ± 7.56 | 15.4 ± 2.84 | 1.27 |
| carboplatin | 7 | 258 ± 86.3 | 227 ± 85.8 | 1.13 |
| oxaliplatin | 7 | 33.0 ± 9.12 | 3.89 ± 1.30 | 8.48*** |
| oxaliplatin | 24 | 14.3 ± 5.55 | 1.79 ± 0.58 | 7.95** |
| oxaliplatin | 72 | 9.64 ± 1.85 | 1.68 ± 0.27 | 5.73*** |

*p < 0.05;
**p < 0.01;
***p < 0.001.

A similar pattern of observations was obtained in human OCT2-transfected cells, but the increase in oxaliplatin cytotoxicity was much more pronounced. As shown in Table 2 and FIG. 2B, the IC₅₀ values of oxaliplatin after different time periods (7, 24, and 72 hr) of exposure were all markedly greater in HEK-MOCK cells than in HEK-OCT2 cells with RF values ranging from 48.4 to 76.7 (p<0.05 to p<0.001). However, the IC₅₀ values of cisplatin and carboplatin were only slightly greater in HEK-MOCK cells than in HEK-OCT2 cells with RF values around 2 after 7-hour drug exposure (Table 2). Co-incubation with an OCT inhibitor, cimetidine (1.5 mM), dramatically increased the oxaliplatin IC₅₀ (control vs. cimetidine-treated: 0.039±0.025 μM vs. 2.81±1.63 μM) by 72-fold (p<0.05) in HEK-hOCT2 cells, with only a 3.18-fold increase in HEK-MOCK cells (control vs. cimetidine-treated: 2.99±1.51 vs. 9.50±2.95 μM, p<0.05) (FIG. 2E). Cimetidine itself did not exhibit cytotoxicity up to a concentration of 5 mM under the same test conditions. These results indicate that OCT2 markedly enhances the cytotoxicity of oxaliplatin with only slight effects on the cytotoxicities of cisplatin and carboplatin.

TABLE 2
Drug sensitivity of cisplatin, carboplatin, and oxaliplatin
in OCT-transfected cells: Cytotoxicity, expressed as IC₅₀, of
the platinum-based drugs in HEK-MOCK and HEK-hOCT2 cells.

| Platinum | Drug Exposure | HEK-MOCK | HEK-hOCT2 | |
|---|---|---|---|---|
| Drugs | Time (hour) | (μM) | (μM) | RF |
| cisplatin | 7 | 2.95 ± 0.23 | 1.32 ± 0.18 | 2.23*** |
| carboplatin | 7 | 110 ± 46.3 | 61.6 ± 46.3 | 1.78 |
| oxaliplatin | 7 | 2.99 ± 1.51 | 0.039 ± 0.025 | 76.7** |
| oxaliplatin | 24 | 1.50 ± 0.69 | 0.02 ± 0.001 | 73.8* |
| oxaliplatin | 72 | 0.93 ± 0.056 | 0.019 ± 0.004 | 48.4*** |

*p < 0.05;
**p < 0.01;
***p < 0.001.

In contrast to OCT1 and OCT2, overexpression of human OCT3 did not affect the cytotoxicity of any of the platinum-based drugs (Table 3 and FIG. 2C).

TABLE 3
Drug sensitivity of cisplatin, carboplatin, and oxaliplatin
in OCT-transfected cells: Cytotoxicity, expressed as IC₅₀, of
the platinum-based drugs in HEK-MOCK and HEK-hOCT3 cells.

| Platinum | Drug Exposure | HEK-MOCK | HEK-hOCT3 | |
|---|---|---|---|---|
| Drugs | Time (hour) | (μM) | (μM) | RF |
| cisplatin | 7 | 2.83 ± 0.90 | 2.44 ± 0.71 | 1.16 |
| carboplatin | 7 | 84.8 ± 9.71 | 48.1 ± 23.4 | 1.76 |
| oxaliplatin | 7 | 1.47 ± 0.28 | 2.22 ± 0.41 | 0.66 |
| oxaliplatin | 24 | 0.47 ± 0.05 | 0.62 ± 0.19 | 0.75 |
| oxaliplatin | 72 | 0.47 ± 0.12 | 0.69 ± 0.19 | 0.68 |

The IC₅₀ values (μM) of cisplatin, carboplatin, and oxaliplatin in human OCT1-, OCT2-, and OCT3-transfected cell lines were determined in parallel with those in the corresponding MOCK cells using MTT assay as described above. Briefly, the cells were seeded in 96-well plates at a density of 5,000 cells/well for the transfected MDCK cells or 12,000 cells/well for the transfected HEK 293 cells. The platinum-based drugs were added on the following day. After the specified time periods of drug exposure, the drug-containing medium was replaced with fresh, drug-free medium, and the incubation was continued for a total of 72 hours (start from the time when the drug was added). After incubation, the cell growth was determined by an MTT assay. Data are expressed as mean±SD from 3 to 6 independent experiments with each performed in quadruplicate. The resistance factor (RF) was defined as the ratio of the mean IC₅₀ value in the MOCK cells to that in the OCT-transfected cells.

### Platinum Accumulation Rates in Cells After Exposure to Cisplatin, Carboplatin, and Oxaliplatin

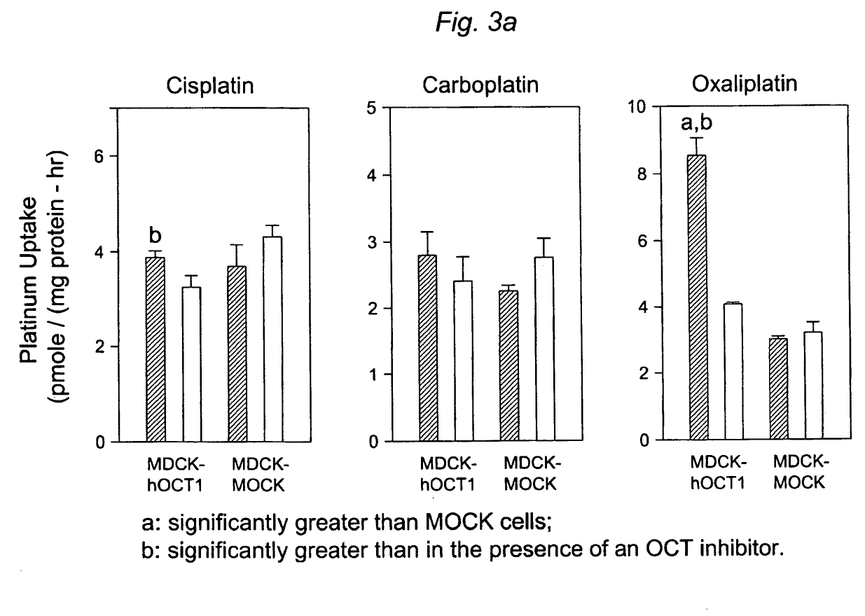

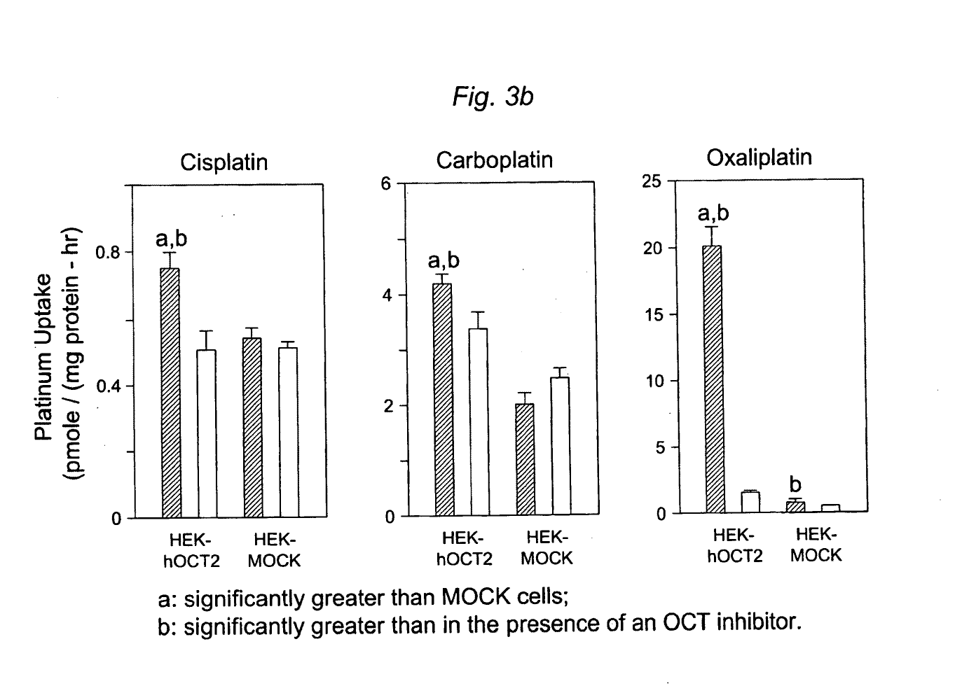

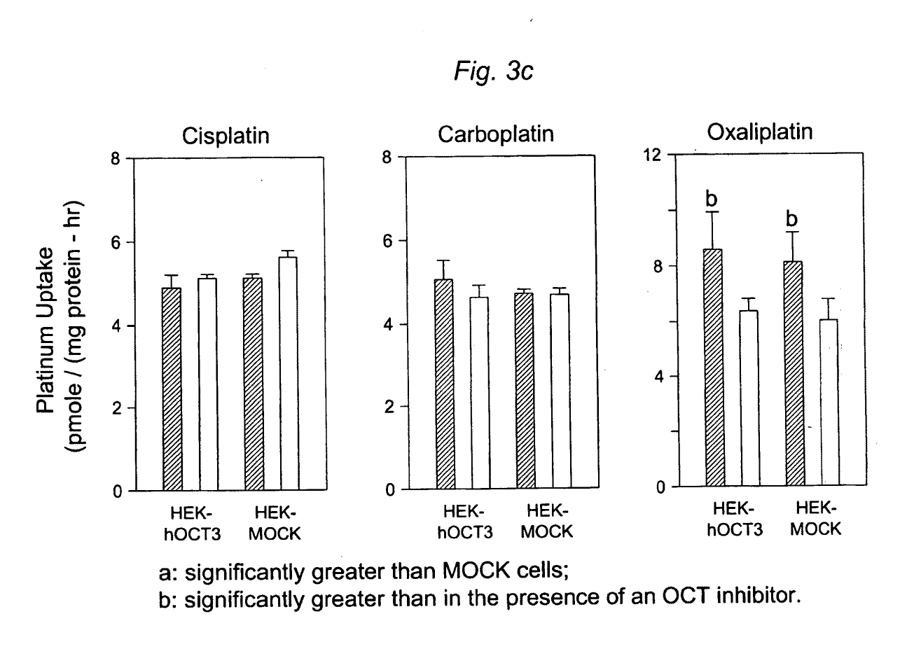

The cellular platinum accumulation rate after two hours of exposure to oxaliplatin (3 μM) was 2.90-fold higher (p<0.001) in MDCK-hOCT1 cells (8.53±0.52 pmol/mg protein-hr) than that in MDCK-MOCK cells (2.94±0.11 pmol/mg protein-hr) (FIG. 3A). Co-incubation with disopyramide (150 μM) resulted in a two-fold decrease in the rate of platinum accumulation in MDCK-hOCT1 cells (control vs. disopyramide-treated: 8.53±0.52 vs. 4.04±0.04 pmol/mg protein-hr, p<0.001) with little effect in MDCK-MOCK cells (control vs. disopyramide-treated: 2.94±0.11 vs. 3.23±0.31 pmol/mg protein-hour, p>0.05) (FIG. 3A). However, the cellular accumulation rates of platinum after 2-hr exposure to cisplatin (3 μM) or carboplatin (15 μM) in MDCK-hOCT1 cells (cisplatin: 3.88±0.15 pmol/mg protein-hr; carboplatin: 2.77±0.36 pmol/mg protein-hr) were not significantly different from those in MDCK-MOCK cells (cisplatin: 3.70±0.45 pmol/mg protein-hr; carboplatin: 2.22±0.07 pmol/mg protein-hr) and were not inhibited by disopyramide (FIG. 3A). These results indicate that human OCT1 contributes substantially to the uptake of oxaliplatin, but not cisplatin or carboplatin in OCT1-transfected cells.

The platinum accumulation rate in HEK-hOCT2 (20.2±1.54 pmol/mg protein-hr) was markedly higher (21.7-fold, p<0.001) than that in HEK-MOCK cells and was substantially reduced in the presence of cimetidine (control vs. cimetidine: 20.2±1.54 vs. 1.80±0.13 pmol/mg protein-hr). However, the cellular accumulation rate of platinum in HEK-hOCT2 cells after 2-hr exposure to cisplatin (0.3 μM) or carboplatin (10 μM) (cisplatin: 0.738±0.055 pmol/mg protein-hr; carboplatin: 4.17±0.18 pmol/mg protein-hr) was only modestly higher (1.38-fold for carboplatin, p<0.001; 2.08-fold for cisplatin, p<0.01) than that in HEK-MOCK cells. Co-incubation with cimetidine (1.5 mM) produced only a small decrease (less than 1.5-fold, p<0.01) in platinum accumulation rate after exposure of HEK-hOCT2 cells to either cisplatin or carboplatin with little effect in HEK-MOCK cells. These results indicate that OCT2 plays an important role in the uptake of oxaliplatin in the transfected cells with a much lower effect on the uptake of cisplatin or carboplatin. In contrast to OCT1 and OCT2, OCT3 overexpression did not affect the uptake of any of these platinum-related drugs (FIG. 3C).Platinum-DNA Adduct Formation After 2-hr

### Exposure to Oxaliplatin

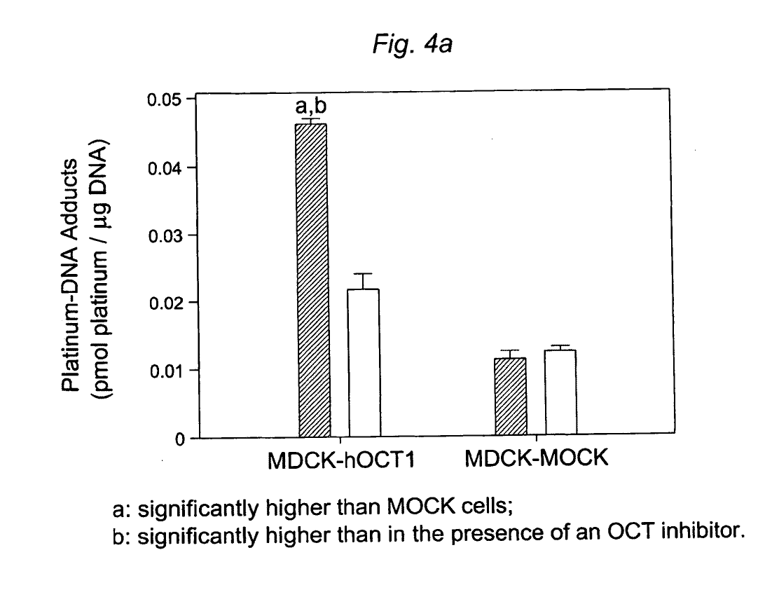

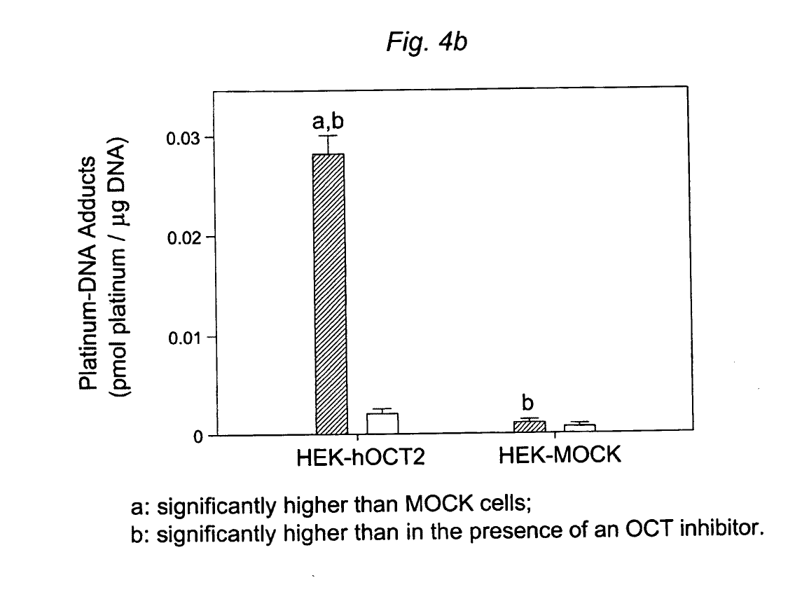

To determine whether the oxaliplatin taken up by cells via the human OCT1 and OCT2 transporters was available for DNA binding, platinum-DNA adduct formation after a 2-hr exposure to oxaliplatin was also measured. FIG. 4A shows that the platinum-DNA adduct level in MDCK-hOCT1 cells (0.0457±0.0011 pmol/μg DNA, r_{b} (ratio of bound platinum atoms pernucleotide)=1.51±0.04×10⁻⁵) was 4.15-fold greater (p<0.001) than that in MDCK-MOCK cells (0.0110±0.0010 pmol/μg DNA, r_{b}=3.63±0.33×10⁻⁶) after exposure to oxaliplatin. Co-incubation with disopyramide (150 μM) significantly decreased (2.11-fold, p<0.001) platinum-DNA adduct formation in MDCK-hOCT1 cells (control vs. disopyramide-treated: 0.0457±0.0011 pmol/μg DNA, r_{b}=1.51±0.04×10⁻⁵ vs. 0.0217±0.0019 pmol/μg DNA, r_{b}=7.16±0.63×10⁻⁶) with no effect in MDCK-MOCK cells. FIG. 4B shows that the platinum-DNA adduct level in HEK-hOCT2 cells (0.0284±0.0020 pmol/μg DNA, r_{b}=9.37±0.66×10⁻⁶) was 28.8-fold higher (p<0.001) than that in HEK-MOCK after exposure to oxaliplatin and was markedly reduced by cimetidine (0.00216±0.00031 pmol/μg DNA, r_{b}=9.37±0.66×10⁻⁶ vs. 7.13±1.02×10⁻⁷). Cimetidine produced only a small decrease (1.70-fold, p<0.05) in HEK-MOCK cells.

### Structure-Activity Relationships (SAR) for Platinum-OCT1 Interaction

To investigate the SAR for platinum-OCT1 interactions, the drug sensitivities (IC₅₀) and RF values of 9 platinum complexes (FIG. 5) in both MDCK-MOCK and MDCK-hOCT1 cells were determined (Table 4). The results described below indicate that the higher the RF value, the higher the interaction between the platinum-based compound and OCT1.

TABLE 4
Drug sensitivity of platinum-based compounds:
Drug sensitivity of structurally diverse platinum
complexes in OCT1-transfected cells.

| | MDCK-MOCK | MDCK-hOCT1 | |
|---|---|---|---|
| Platinum Complexes | (μM) | (μM) | RF |
| cisplatin | 6.32 ± 0.74 | 3.58 ± 0.30 | 1.76** |
| carboplatin | 258 ± 86.3 | 227 ± 85.8 | 1.13 |
| [Pt(NH₃)₂(trans- | 21.4 ± 2.94 | 10.8 ± 2.66 | 1.97*** |
| 1,2-(OCO)₂C₆H₁₀] | | | |
| [Pt(Cl₂)(en)] | 33.2 ± 11.5 | 10.2 ± 4.76 | 3.26** |
| cis-[Pt(NH₃)(Cy)Cl₂] | 1.42 ± 0.15 | 0.16 ± 0.03 | 9.02*** |
| oxaliplatin | 10.9 ± 3.66 | 0.48 ± 0.19 | 22.4*** |
| [Pt(S,S-DACH)oxalato] | 30.0 ± 14.2 | 1.45 ± 1.16 | 20.7*** |
| [Pt(R,R-DACH)Cl₂] | 15.0 ± 3.24 | 0.65 ± 0.26 | 22.9*** |
| [Pt(S,S-DACH)Cl₂] | 16.2 ± 3.72 | 0.57 ± 0.18 | 28.4*** |

**p < 0.01;
***p < 0.001.

Nature of the non-leaving group(s): RF values less than two were obtained for platinum complexes with diammine non-leaving groups including cisplatin, carboplatin, and [Pt(NH₃)₂(trans-1,2-(OCO)₂C₆H₁₀], indicating that platinum-based compounds with this purely inorganic non-leaving unit are poorly recognized by OCT1 (Table 4). However, when the non-leaving group(s) contained an organic component as in [PtCl₂(en)], which has two methylene groups between the amine functionalities, the RF value increased to 3.26. Moreover, with increasing size of the organic component of the non-leaving group(s), the interaction of a platinum-based compound with OCT1 increased. For example, the platinum-based compounds cis-[Pt(NH₃)(Cy)Cl₂], the R,R- and S,S-isomers of oxaliplatin and [Pt(DACH)Cl₂], which all have a 6-C cyclohexyl moiety as part of their non-leaving group, had high RF values (9.02-28.4) (Table 4). Therefore, the structure of the non-leaving group(s) of a platinum-based compound is an important determinant of its interaction with OCT1. Lastly, different isomers of the 1,2-diaminocyclohexane-substituted platinum complexes interact similarly with OCT1. For example, the R,R- and S,S-isomers of oxaliplatin (R,R vs. S,S: 22.4 vs. 20.7) and [Pt(DACH)Cl₂] (R,R vs. S,S: 22.9 vs. 28.4) have similar RF values (Table 4).

Nature of the leaving group(s): Changes in the leaving group did not induce substantial changes in the RF values of platinum complexes. For example, all the DACH compounds (R,R- and S,S-isomers of oxaliplatin and [Pt(DACH)Cl₂]) had similar RF values (20.7-28.4; Table 4), although the leaving group of oxaliplatin (oxalate) is very different from that of [Pt(DACH)Cl₂] (chloride). In addition, cisplatin, carboplatin, and [Pt(NH₃)₂(trans-1,2-(OCO)₂C₆H₁₀], all of which have different leaving groups but identical non-leaving groups, had similar RF values (1.13-1.97; Table 4). Moreover, a cyclohexane ring, when present in the non-leaving group(s) of a platinum complex, such as those in DACH compounds, markedly increases OCT1 interaction (RF: 20.7-28.4) in comparison to diammine ligands (RF: 1.13-1.97). However, when the cyclohexane ring was incorporated into the leaving group, as in [Pt(NH₃)₂(trans-1,2-(OCO)₂C₆H₁₀], it had no effect on the OCT1 interaction, the RF value of [Pt(NH₃)₂(trans-1,2-(OCO)₂C₆H₁₀] being 1.97 (Table 4).

The IC₅₀ values (μM) of all 9 platinum complexes, except for carboplatin, in MDCK-MOCK and MDCK-hOCT1 after 7 hours of drug exposure were determined in parallel using an MTT assay as described above. Briefly, MDCK cells were seeded at a density of 5,000 cells/well in 96-well plates and exposed to the test compounds for 7 hours on the following day. After incubation for a total of 72 hours, the cell growth was determined by an MTT assay. The data for carboplatin was taken from Table 1 and was not determined simultaneously with the other compounds. The resistance factor (RF) was defined as the ratio of the mean IC₅₀ value in MDCK-MOCK cells to that in MDCK-hOCT1 cells. Data are expressed as mean±SD from six measurements, and each measurement was performed in quadruplicate.

Identification of the Chemical Form of Oxaliplatin that is the Substrate(s) of OCT1

Multiple chemical species exist in equilibrium when platinum complexes are dissolved in an aqueous solution containing high concentrations of chloride ion (Desoize et al., (2002); Howe-Grant et al., (1980)). Therefore, identification of the chemical species that are taken up by OCT1 would contribute to an understanding of the SAR of platinum-OCT1 interactions. In chloride containing media, such as plasma ([Cl⁻]˜103 mM (Howe-Grant et al., supra)) and the cell culture medium used in the present study, the oxalate leaving group of oxaliplatin can be replaced by chloride, resulting in [Pt(R,R-DACH)Cl₂]. The latter can be further aquated to form the mono-, [Pt(R,R-DACH)(H₂O)Cl]⁺, and dicationic, [Pt(R,R-DACH)(H₂O)₂]²⁺, species Di Francesco et al., (2002)). The monoaqua and diaqua cations are the active forms of oxaliplatin, which bind to DNA. Considering the general properties of OCT substrates, which are positively charged small organic compounds, it is likely that the mono- and/or diaqua chemical species, having one or two positive charges, are the chemical forms taken up by OCT1.

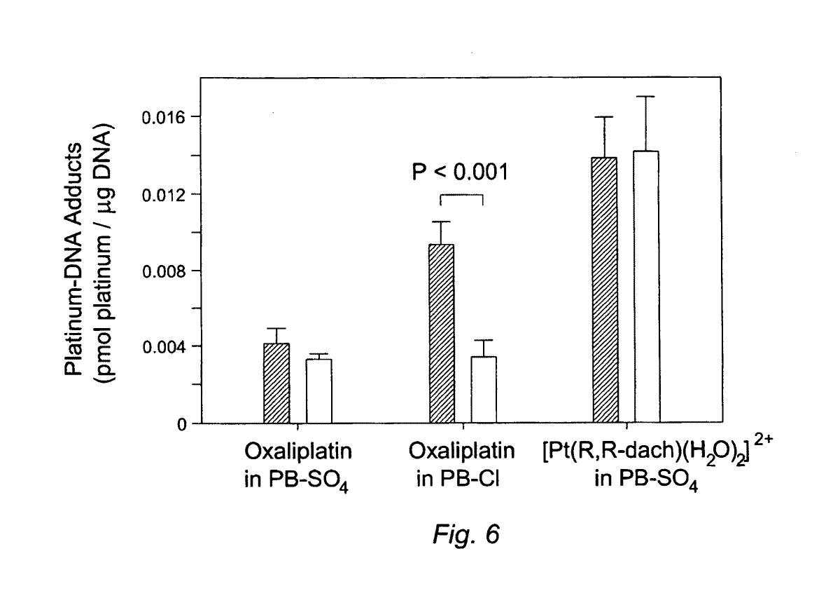

To investigate experimentally the oxaliplatin-derived species taken up by OCT1, platinum-DNA adduct formation in both MDCK-hOCT1 and MDCK-MOCK cells after incubation with oxaliplatin (20 μM) in chloride free buffer (PB—SO₄) was first measured. In this buffer, oxaliplatin should remain predominantly intact because the affinity of sulfate for platinum(II) is much lower than that of chloride (Howe-Grant et al., supra). Displacement of the oxalate group by water will be a relatively slow process. In addition, short incubation times (25 min) were used to minimize conversion of oxaliplatin to intermediate aquated species. Under these conditions, the Pt-DNA adduct level in MDCK-hOCT1 cells (0.00398±0.00089 pmol/μg DNA, r_{b}=1.31±0.29×10⁻⁶) was similar to (p>0.05) that in MDCK-MOCK cells (0.00320±0.00042 pmol/μg DNA, r_{b}=1.05±0.14×10⁻⁶) (FIG. 6), indicating that unmodified oxaliplatin is not an OCT1 substrate. Secondly, to determine whether an aquated form of oxaliplatin was taken up by OCT1, platinum-DNA adduct formation was measured after incubation with oxaliplatin (20 μM) in the chloride-containing buffer, PB—Cl, for 25 min. Under these conditions, it is likely that conversion to the monochloro/monoaqua cation occurs, with displacement of the oxalate ligand. The DNA-associated platinum level was substantially higher (2.74-fold, p<0.01) in MDCK-hOCT1 cells (0.00933±0.00124 pmol/μg DNA, r_{b}=3.08±0.41×10⁻⁶) than that in MDCK-MOCK cells (0.00340±0.00087 pmol/μg DNA, r_{b}=1.12±0.29×10⁻⁶) (FIG. 6), consistent with this expectation. Platinum-DNA adduct formation after direct incubation with the diaqua compound, [Pt(R,R-DACH)(H₂O)₂]^{2+ (}1 μM), in the PB—SO₄ buffer for 25 min was also determined. Under these conditions, the platinum complex is a mixture of diaqua (82.8%) and aqua/hydroxo (17.1%) species. Here, the percentage was calculated based on the pKa values of 6.14 and 7.56 for the diaqua and aqua/hydroxo forms of oxaliplatin, respectively (Gill et al., J. Am. Chem. Soc., 104:4598-4604 (1982)), and the pH value of 7.4 for the incubation buffer. The DNA-associated platinum level in MDCK-hOCT1 cells (0.0139±0.0020 pmol/μg DNA, r_{b}=4.59±0.66×10⁻⁶) was similar to (p>0.05) that in MDCK-MOCK cells (0.0142±0.0028 pmol/μg DNA, r_{b}=4.69±0.92×10⁻⁶) (FIG. 6), indicating that the diaqua form is not an OCT1 substrate. Whether or not the aqua/hydroxo form, which carries one positive charge, can be taken up by OCT1 remains unclear. Taken together, these studies illustrate that a monoaquated form of oxaliplatin, either the chloro or hydroxo species, both of which carry one positive charge, is the actual substrate of OCT1.

### Expression of OCT1 and OCT2 in Colon Cancer Cell Lines and Tissue Samples

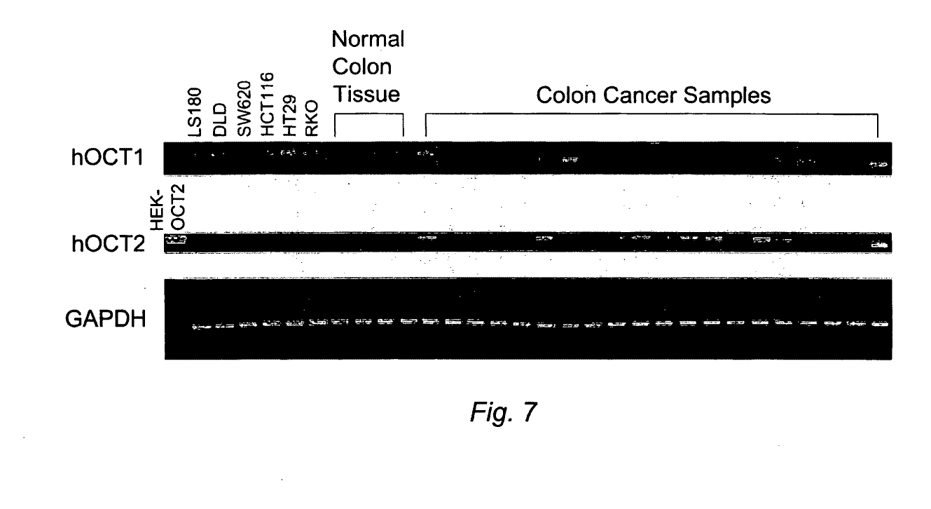

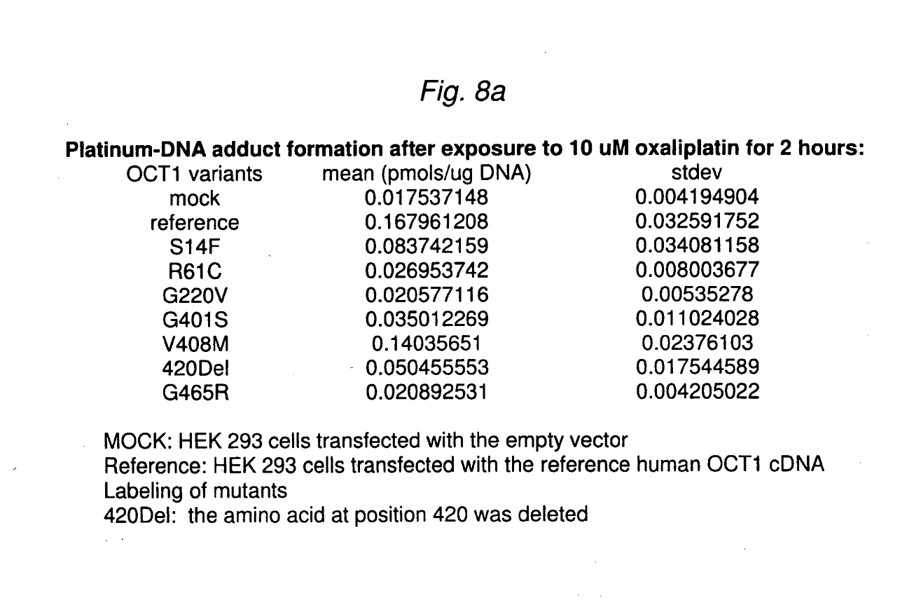

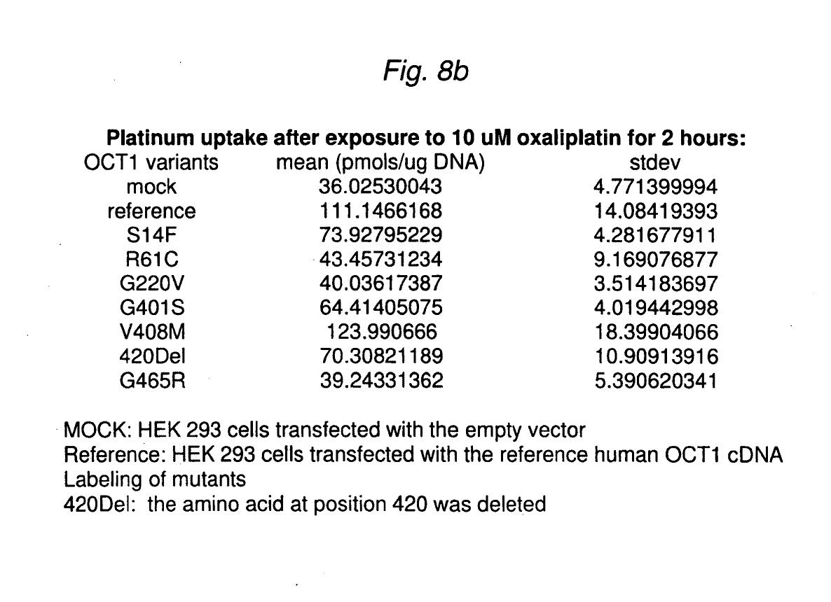

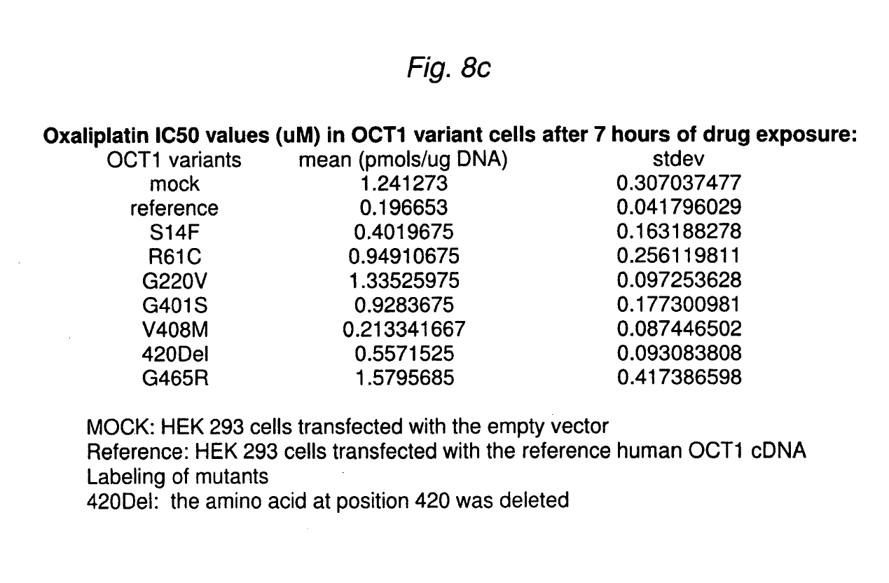

Since oxaliplatin is currently approved for advanced colon cancer therapy, the expression of OCT1 and OCT2 in colon cancer cell lines and tumor samples was determined. As shown in FIG. 7, expression of OCT1 mRNA was detected in the six colon cancer cell lines tested in this study (LS180, DLD, SW620, HCT116, HT29, and RKO) with the highest expression level in HT29 cells. Four normal colon tissue samples and twenty colon tumor samples exhibited variable OCT1 expression levels. OCT2 was not detected in any of the cell lines or in the normal colon tissue samples; however, 11 of the 20 tumor samples demonstrated significant OCT2 expression (FIG. 7).

### The Effect of an OCT Inhibitor, Cimetidine, on Drug Sensitivity of Cisplatin and Oxaliplatin in Colon Cancer Cell Lines

To evaluate the potential role of OCT1 in the cytotoxicity of oxaliplatin and to determine whether OCT1 contributes to the differences in activities of cisplatin and oxaliplatin, the sensitivities (IC₅₀) of both oxaliplatin and cisplatin in the colon cancer cells was determined in the presence and absence of an OCT inhibitor, cimetidine (1.5 mM). The resistance factor (RF) due to the presence of cimetidine was defined as the ratio of the IC₅₀ value in the presence of cimetidine to that in the absence of cimetidine. As shown in Table 5, the sensitivity of oxaliplatin was higher (lower IC₅₀) than that of cisplatin in each of the tested colon cancer cell lines in the absence of cimetidine (control, the mean±SE of IC₅₀ in the six cell lines: 3.88±1.42 μM (oxaliplatin) vs. 10.5±2.02 μM (cisplatin)). However, in the presence of cimetidine, oxaliplatin sensitivity was substantially decreased in each of the cell lines (RF values ranged from 5.04 to 11.4 (p<0.001)), resulting in IC₅₀ values comparable to, or even higher than those of cisplatin (mean±SE of IC₅₀ in the six cell lines: 29.1±10.7 μM (oxaliplatin) vs. 19.4±4.32 μM (cisplatin)). The effect of cimetidine on cisplatin sensitivity was small (range of RF values: 1.44-2.47, Table 5).

TABLE 5
Drug sensitivity of platinum-based compounds: The sensitivity of the colon cancer
cell lines to oxaliplatin and cisplatin in the presence and absence of cimetidine.

| | Oxaliplatin | | | Cisplatin | | |
|---|---|---|---|---|---|---|
| | | Cimetidine- | | | Cimetidine- | |
| Cell Lines | Control | treated | RF | Control | treated | RF |
| HCT116^{a} | 2.37 ± 1.44 | 18.8 ± 6.19 | 7.93*** | 5.42 ± 1.34 | 10.2 ± 3.23 | 1.88** |
| HT29^{a} | 4.56 ± 1.40 | 52.1 ± 18.5 | 11.4*** | 12.4 ± 3.91 | 30.8 ± 11.0 | 2.47** |
| RKO^{a} | 1.64 ± 0.56 | 9.70 ± 2.70 | 5.92*** | 8.58 ± 2.38 | 12.5 ± 4.44 | 1.46 |
| SW620^{a} | 2.81 ± 1.00 | 14.2 ± 2.81 | 5.04*** | 12.6 ± 1.95 | 22.2 ± 4.92 | 1.76** |
| LS180^{a} | 1.30 ± 0.41 | 8.39 ± 2.77 | 6.44*** | 5.72 ± 1.75 | 8.27 ± 3.35 | 1.44 |
| DLD | 10.6 ± 5.99 | 71.3 ± 12.9 | 6.73*** | 18.4 ± 7.58 | 32.3 ± 10.3 | 1.74* |

*p < 0.05;
**p < 0.01;
***p < 0.001.
^{a}The IC₅₀ value of oxaliplatin is significantly lower than that of cisplatin in the absence of cimetidine.

The IC₅₀ values (μM) of oxaliplatin and cisplatin in the colon cancer cell lines were determined in the presence or absence (control) of cimetidine (1.5 mM) as described above. The cell seeding density was 6,000-, 8,000-, 6,000-, 15,000-12,000- and 4,000 cells/well for HCT116, HT29, RKO, SW620, LS180, and DLD cells, respectively. When cimetidine (1.5 mM) was used, it was added to the wells immediately before the addition of the platinum-based drugs. The resistance factor (RF) was defined as the ratio of the mean IC₅₀ value in the presence to that in the absence of cimetidine. Data are expressed as mean±SD from six measurements, and each measurement was performed in quadruplicate.

### Discussion

The striking activity of cisplatin in an otherwise fatal disease, testicular cancer, has been established by thirty years of clinical experience. However, acquired and intrinsic resistance limits its application to a relatively narrow range of tumor types. To broaden the anticancer spectrum of this platinum agent, thousands of structural analogs have been tested. Cisplatin analogs with two ammine ligands, such as carboplatin and nedaplatin (approved in Japan), are cross-resistant with cisplatin (Lebwohl et al., Eur. J. Cancer, 34:1522-1534 (1998)). Analogs with different ligands display more diverse activity profiles (Rixe et al., Biochem. Pharmacol., 52:1855-1865 (1996)). Notably, oxaliplatin, with DACH in place of the two amine ligands, in combination with 5-fluoruracil/leucovorin, produced response rates twice that of 5-fluoruracil/leucovorin regimens alone in the treatment of colorectal cancer (Kelly et al., J. Clin. Oncol., 23:4553-4560 (2005)), against which cisplatin is inactive (Misset et al., Crit. Rev. Oncol. Hematol., 35:75-93 (2000)). Efforts to understand the differences in oxaliplatin versus cisplatin antitumor activity have focused mainly on the cellular processing of cisplatin- and oxaliplatin-DNA adducts (Chaney et al., Crit. Rev. Oncol. Hematol., 53:3-11 (2005); Fink et al., Cancer Res., 56:4881-4886 (1996); Vaisman et al., Biochemistry, 38:11026-11039 (1999)). Defects in MMR cause modest to moderate resistance to cisplatin but not to oxaliplatin (Fink et al., supra; Fink et al., Cancer Res., 57:1841-1845 (1997)). Differences in the mechanism(s) controlling cellular uptake and efflux of these platinum-based compounds, although rarely studied, can also contribute to their disparate activities considering the nature of their chemical structures.

The present study demonstrates that the influx transporters, OCT1 and OCT2, play an important role in the cellular uptake and consequent cytotoxicity of oxaliplatin (Tables 1-2 and FIG. 2). In contrast, these two transporters were relatively unimportant in mediating the uptake and cytotoxicity of cisplatin and carboplatin. Overexpression of OCT1 and, more strikingly, OCT2 in transfected cells not only increased the rate of cellular platinum accumulation but also elevated the level of platinum-DNA adducts after oxaliplatin exposure (FIGS. 3 and 4). These effects were blocked by known OCT inhibitors. The data strongly indicate that oxaliplatin is an excellent substrate of human OCT1 and OCT2, and the cellular uptake of platinum mediated by these transporters has ready access to the key pharmacological target, DNA. These results are in contrast to platinum uptake mediated by human Ctr1, which appears to sequester the drug in some intracellular compartment, rendering it inaccessible to the pharmacological target (Holzer et al., Mol. Pharmacol., 66:817-823 (2004)). A modest increase in cisplatin uptake (FIG. 3B) and sensitivity (2.23-fold, p<0.001, Table 2) was observed in HEK-hOCT2 cells in comparison to HEK-MOCK cells, indicating that cisplatin is a weak substrate of OCT2.

It is noteworthy that expression of OCT1 or OCT2, even at low levels, may play a significant role in the cytotoxicity of oxaliplatin. A more than three-fold increase (3.18-fold) in the IC₅₀ value of oxaliplatin in HEK-MOCK cells was consistently observed in the presence of the OCT inhibitor, cimetidine (FIG. 2E), but not for cisplatin or carboplatin. The decrease in oxaliplatin sensitivity in HEK-MOCK cells by the OCT inhibitor is most likely due to inhibition of intrinsic OCT1 and/or OCT2 activity in HEK 293 cells. Both transporters were detected in HEK-MOCK cells in PCR studies using a cycle number of 40. Furthermore, cimetidine consistently produced a significant decrease in the cellular uptake of oxaliplatin, but not of cisplatin or carboplatin in HEK-MOCK cells (FIGS. 3B and 3C). Although it might be possible that cimetidine reacts with the platinum compounds and therefore inactivates them, this explanation is unlikely to be of primary importance since similar effects of cimetidine on the cellular uptake and cytotoxicity of cisplatin and carboplatin would have been expected. Taken together, the data indicate that low levels of expression of OCT1 and OCT2 play a significant role in sensitizing cells to oxaliplatin.

Structure-activity relationship studies revealed that the nature of the amine ligand bound to platinum is important for interaction with OCT1, with an organic component being required for effective interaction. On the other hand, the structure of the leaving ligand seems to be unimportant. A monoaqua derivative of oxaliplatin, specifically the monoaqua/monochloride species, and not a divalent diaqua complex, is likely to be the preferred substrate of OCT1 (FIG. 6). These results are consistent with previous work showing that OCTs interact with small molecular weight monovalent organic cations (Jonker et al., Pharmacol. Exp. Ther., 308:2-9 (2004)). Although the structure-activity relationships were established for platinum-OCT1 interactions, it is likely that the conclusions apply to platinum-OCT2 interactions because the two transporters have largely overlapping substrate specificities. These studies establish the basis for the design of additional platinum complexes to facilitate the development of an even more detailed structure-activity relationship, which could be used to predict their interaction with OCTs. These studies also illustrate the potential to target platinum complexes for therapy against tumors that express OCT1 and OCT2.

The structure-activity relationship studies further suggest that OCTs do not play a major role in determining the cytotoxicity of platinum-based compounds with two amine ligands, such as cisplatin, carboplatin, and nedaplatin. In contrast, OCTs may be important for mediating cytotoxicity of platinum-based compounds with organic amine ligands (Table 4). Cell lines that are resistant to cisplatin are cross-resistant to the diammine complexes, carboplatin and nedaplatin, but not to the DACH compounds, oxaliplatin and tetraplatin, which share a similar activity profile (Lebwohl et al., supra; Rixe et al., supra). Differences in the activity profiles of these compounds parallel the differences in their interaction with OCTs, suggesting that interactions with OCT1 and OCT2 may explain, at least in part, differences in the activities and tumor specificities of platinum complexes.

It is likely that the activity of oxaliplatin in colorectal cancer can be explained, at least in part, by the selective uptake via OCTs. In this study, OCT1 expression was detected in all twenty human colon cancer tissue samples and OCT2 expression in 11 out of 20 tissue samples (FIG. 7). Similar levels of OCT1 were also detected in the six tested human colon cancer cell lines, although OCT2 was not detectable. However, both OCT1 and OCT2 expression have been detected in another human colon cancer cell line, Caco-2 (Hayer-Zillgen et al., Br. J. Pharmacol., 136:829-836 (2002); Muller et al., Biochem. Pharmacol., 70:1851-1860 (2005)). Drug sensitivity to oxaliplatin was greater than that of cisplatin in each of the six colon cancer cell lines (Table 5). The higher activity of oxaliplatin in comparison to that of cisplatin in these colon cancer cells is a consequence of the selective uptake of oxaliplatin mediated by the intrinsic OCT1 in these cells, since similar activities of oxaliplatin and cisplatin were observed in these cells when OCT1 was blocked by cimetidine.

Based on the expression of OCT1 and OCT2 in the colon cancer tissue samples and the OCT-dependent activity of oxaliplatin in the cell lines, the present study demonstrates that these transporters are important determinants of oxaliplatin activity in colorectal cancer. Also, variable expression of OCTs, especially OCT2, may account for the variability in response to oxaliplatin treatment. As a result, the expression levels of OCT1 and OCT2 may be used as markers for the rational selection of oxaliplatin-based versus irrinotecan-based combination therapies for treatment of individuals with colorectal cancer. Such selection is now primarily based on side-effect profiles or clinical experience (Goldberg, Oncologist, 10 Suppl 3:40-48 (2005)). Oxaliplatin-based therapy may be a better choice for patients with high levels of OCT1 and OCT2 in their tumor samples. In addition, genotyping for non-functional and reduced function polymorphisms of OCT1 and OCT2 may be incorporated in the diagnostic or prognostic process.

Currently, platinum-based therapies are used in the treatment of a variety of tumors including testicular cancer, ovarian cancer, small cell lung cancer, and head and neck cancers (Lebwohl et al., supra). In these therapies, cisplatin is often the drug of choice because other platinum-based compounds such as oxaliplatin are not superior. However, the present study indicates that when OCT1 or OCT2 is expressed in the tumor, oxaliplatin is a better choice. The present study also suggests that in addition to efflux transporters (Szakacs et al., Cancer Cell, 6:129-137 (2004)), influx transporters play a significant role in determining tumor sensitivity/resistance to anticancer agents (Huang et al., Cancer Res., 64:4294-4301 (2004)). Recently, OCT1 and OCT2 expression has been observed in a number of human cancer cell lines (Hayer-Zillgen et al., supra), indicating that these transporters may be expressed in the corresponding tumors. The results of this study clearly illustrate that determining the expression of one or more OCTs in a tumor sample can provide a basis for the rationale selection of platinum-based therapies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of providing a prognosis for oxaliplatin cancer therapy in a subject, the method comprising the steps of:
(a) contacting a sample from the subject with an antibody that specifically binds to OCT protein; and
(b) determining whether or not OCT protein is expressed in the sample, thereby providing a prognosis for oxaliplatin cancer therapy.

2. The method of claim 1, wherein the cancer is selected from the group consisting of colorectal cancer, liver cancer, prostate cancer, renal cancer, bladder cancer, ovarian cancer, breast cancer, lung cancer, leukemia, non-Hodgkin's lymphoma, and multiple myeloma.

3. The method of claim 1, wherein the OCT is selected from the group consisting of OCT1, OCT2, OCT3, and combinations thereof.

4. The method of claim 1, wherein the sample is from colon, rectum, liver, kidney, bladder, prostate, ovary, bone, or lymph node.

5. The method of claim 1, wherein the antibody is a monoclonal or polyclonal antibody.

6. The method of claim 1, wherein the method further comprises genotyping the subject to determine an OCT genotype.

7. A method of providing a prognosis for oxaliplatin cancer therapy in a subject, the method comprising the steps of:
(a) contacting a sample from the subject with a primer set of a first oligonucleotide and a second oligonucleotide that each specifically hybridize to an OCT nucleic acid;
(b) amplifying the OCT nucleic acid in the sample; and
(c) determining whether or not the OCT nucleic acid in the sample is expressed in the sample, thereby providing a prognosis for oxaliplatin cancer therapy.

8. The method of claim 7, wherein the cancer is selected from the group consisting of colorectal cancer, liver cancer, prostate cancer, renal cancer, bladder cancer, ovarian cancer, breast cancer, lung cancer, leukemia, non-Hodgkin's lymphoma, and multiple myeloma.

9. The method of claim 7, wherein the OCT is selected from the group consisting of OCT1, OCT2, OCT3, and combinations thereof.

10. The method of claim 7, wherein the sample is from colon, rectum, liver, kidney, bladder, prostate, ovary, bone, or lymph node.

11. The method of claim 7, wherein the first oligonucleotide comprises SEQ ID NO:1 and the second oligonucleotide comprises SEQ ID NO:2.

12. The method of claim 7, wherein the first oligonucleotide comprises SEQ ID NO:3 and the second oligonucleotide comprises SEQ ID NO:4.

13. The method of claim 7, wherein the first oligonucleotide comprises SEQ ID NO:5 and the second oligonucleotide comprises SEQ ID NO:6.

14. The method of claim 7, wherein the method further comprises genotyping the subject to determine an OCT genotype.

15. A method of providing a prognosis for oxaliplatin cancer therapy in a subject by determining the genotype of an OCT gene, the method comprising the steps of:
(a) contacting a sample from the subject with an antibody that specifically binds to an OCT protein encoded by a selected allele; and
(b) determining whether or not the OCT protein is expressed in the sample, thereby providing a prognosis for oxaliplatin cancer therapy.

16. The method of claim 15, wherein the cancer is selected from the group consisting of colorectal cancer, liver cancer, prostate cancer, renal cancer, bladder cancer, ovarian cancer, breast cancer, lung cancer, leukemia, non-Hodgkin's lymphoma, and multiple myeloma.

17. The method of claim 15, wherein the OCT is selected from the group consisting of OCT1, OCT2, OCT3, and combinations thereof.

18. The method of claim 15, wherein the OCT genotype is selected from the group consisting of: wild-type OCT, G401S, 420 del, S14F, R61C, G220V, V408M, and G465R.

19. The method of claim 15, wherein presence of the wild-type or V408M variant predicts a better response to oxaliplatin therapy than the presence of the other variants.

20. The method of claim 15, wherein the sample is from colon, rectum, liver, kidney, bladder, prostate, ovary, bone, or lymph node.

21. The method of claim 15, wherein the antibody is a monoclonal or polyclonal antibody.

22. The method of claim 15, wherein the method further comprises genotyping the subject to determine an OCT genotype.

23. A method of providing a prognosis for oxaliplatin cancer therapy in a subject by determining an OCT genotype, the method comprising the steps of:
(a) contacting a sample from the subject with a primer set of a first oligonucleotide and a second oligonucleotide that each specifically hybridize to an OCT allele;
(b) determining whether or not the OCT allele is expressed in the sample, thereby providing a prognosis for oxaliplatin cancer therapy.

24. The method of claim 23, wherein the cancer is selected from the group consisting of colorectal cancer, liver cancer, prostate cancer, renal cancer, bladder cancer, ovarian cancer, breast cancer, lung cancer, leukemia, non-Hodgkin's lymphoma, and multiple myeloma.

25. The method of claim 23, wherein the OCT is selected from the group consisting of OCT1, OCT2, OCT3, and combinations thereof.

26. The method of claim 23, wherein the OCT genotype is selected from the group consisting of: wild-type OCT, G401 S, 420 del, S14F, R61C, G220V, V408M, and G465R.

27. The method of claim 23, wherein presence of the wild-type or V408M variant predicts a better response to oxaliplatin therapy than the presence of the other variants.

28. The method of claim 23, wherein the sample is from colon, rectum, liver, kidney, bladder, prostate, ovary, bone, or lymph node.

29. The method of claim 23, wherein the first oligonucleotide comprises SEQ ID NO:1 and the second oligonucleotide comprises SEQ ID NO:2.

30. The method of claim 23, wherein the first oligonucleotide comprises SEQ ID NO:3 and the second oligonucleotide comprises SEQ ID NO:4.

31. The method of claim 23, wherein the first oligonucleotide comprises SEQ ID NO:5 and the second oligonucleotide comprises SEQ ID NO:6.

32. The method of claim 23, wherein the method further comprises genotyping the subject to determine an OCT genotype.

33. A method of localizing a cancer that expresses an organic cation transporter (OCT) in vivo, the method comprising the step of imaging in a subject a cell expressing OCT, thereby localizing the cancer in vivo.

34. The method of claim 33, wherein the cancer that expresses the OCT is selected from the group consisting of colorectal cancer, liver cancer, prostate cancer, renal cancer, bladder cancer, ovarian cancer, breast cancer, lung cancer, leukemia, non-Hodgkin's lymphoma, and multiple myeloma.
